(19) United States
(12) Patent Application Publication
(10) Pub. No.: US 2026/0258396 A1
(43) Pub. Date: Sep. 3, 2026

(54) METHODS AND COMPOSITIONS FOR CAPTURING NON-CANONICAL CFNA FRAGMENTS

(71) Applicant: Aqtual, Inc., Hayward, CA (US)

(72) Inventors: Diana ABDUEVA, Orinda, CA (US); Katharine DILGER, Redwood City, CA (US); Rachael CUNNINGHAM, Redwood City, CA (US); Kevin LAI, Fremont, CA (US)

(21) Appl. No.: 19/454,197

(22) Filed: Jan. 20, 2026

Related U.S. Application Data
(63) Continuation of application No. PCT/US2024/038879, filed on Jul. 19, 2024.
(60) Provisional application No. 63/514,941, filed on Jul. 21, 2023.

(51) Int. Cl. C12N 15/10; C12Q 1/6806

(52) U.S. Cl. CPC C12N 15/1003; C12Q 1/6806

(57) ABSTRACT

Disclosed herein are improved methods and compositions for facilitating capture of cfNA.

### CROSS-REFERENCE

This application is a continuation of International Application No. PCT/US2024/038879, filed Jul. 19, 2024, which claims the benefit of U.S. Provisional Application No. 63/514,941, filed Jul. 21, 2023, each of which is incorporated by reference herein in its entirety.

### BACKGROUND

Cell free nucleic acids (cfNA), such as but not limited to cfDNA and cfRNA are useful for indicating conditions in a less invasive manner. For example, cfNAs are used in prenatal testing. The use of cfNAs in prenatal testing may be used to determine chromosomal disorders of the fetus.

### SUMMARY

In some aspects, the present disclosure provides for a method of enriching cell-free nucleic acid (cfNA) fragments comprising active chromatin, comprising: (a) contacting a composition comprising said cfNA fragments in solution with an anionic solid surface in the presence of a chaotropic agent, (b) removing unbound cfNA not enriched for active chromatin from said anionic solid surface; and (c) obtaining cfNA bound to said anionic solid surface, thereby enriching said cfNA fragments comprising active chromatin, wherein said method does not involve the use of an biomolecule binding agent directed against said active chromatin. In some embodiments, said cfNA fragments comprising active chromatin comprise at least 1000 nucleotides in length, at least 400 nucleotides in length, or at least 300 nucleotides in length. In some embodiments, said solution is configured to favor capture of cfNA fragments of at least 1000 bp in length or 300 bp in length on said anionic solid surface. In some embodiments, a pH of said composition is less than about 6.5, 6.0. In some embodiments, said solution comprises at least about 3.5% to about 11% alcohol. In some embodiments, said chaotropic agent comprises a guanidinium salt or said composition comprises between 1.5 M and 2 M of said guanidinium salt. In some embodiments, said method enriches said cfNA fragments comprising active chromatin by at least 7-fold compared to cfNA fragments of less than 300 bp. In some embodiments, (b) further comprises washing said anionic solid surface with a wash solution. In some embodiments, said wash solution comprises less than or equal to 30%, 25%, 20%, 15%, 10%, or 5% alcohol. In some embodiments, said wash solution displays a pH of greater than or equal to about 6.5 or 6.0. In some embodiments, said wash solution comprises a nonionic surfactant. In some embodiments, the method further comprises eluting said cfNA fragments comprising active chromatin bound to said anionic solid surface. In some embodiments, said anionic solid surface is a surface of a bead or nanoparticle. In some embodiments, a particle size of said bead or nanoparticle is about 200 nm to about 600 nm. In some embodiments, a particle size of said bead or nanoparticle is about 330 nm to about 490 nm. In some embodiments, a particle size of said bead or nanoparticle is less than about 600 nm, 490 nm, 330 nm, or 200 nm, or any range between these values. In some embodiments, a particle size of said bead or nanoparticle is at most 600 nm, 490 nm, 330 nm, or 200 nm, or any range between these values. In some embodiments, said anionic solid surface comprises silicon dioxide (SiO₂). In some embodiments, said bead or nanoparticle comprises a hydroxyl-derivatized surface layer and a ferric core. In some embodiments, said composition comprising said cfNA fragments in solution is plasma. In some embodiments, said plasma is prepared by low-speed centrifugation of whole blood to separate red blood cells from a first supernatant, transferring said first supernatant to a new consumable vessel, and high-speed centrifugation of said first supernatant to separate said plasma from white blood cells and plasma. In some embodiments, said low speed centrifugation comprises a relative centrifugal force (RCF) of equal to or less than about 300 x g. In some embodiments, said high speed centrifugation comprises an RCF of equal to or greater than 5000 x g. In some embodiments, said method further comprises digesting said plasma with a proteolytic enzyme. In some embodiments, said proteolytic enzyme is Proteinase K. In some embodiments, said method results in an enrichment of said cfNA comprising active chromatin of at least about 7- to 10-fold relative to cfNA not comprising active chromatin. In some embodiments, said cfNA fragments comprising active chromatin comprise cfDNA.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example experimental workflow for cfNA capture favoring non-canonical fragments. Shown is a diagram illustrating the generic operations according to the workflow.

FIG. 2 illustrates improvement in relative non-canonical cfNA fragment capture for the methods described herein versus a commercial cfNA capture method. Shown is an electropherogram trace of size (in bp) versus nucleic acid abundance (measured in RFU) performed on plasma nucleic acids illustrating of intensity and size of cfNA extracted using a commercial protocol (red) and the methods described herein (black).

FIG. 3 illustrates improvement in relative non-canonical cfNA fragment capture for the methods described herein versus a commercial cfNA capture method following library preparation. Shown is an electropherogram trace of size (in bp) versus nucleic acid abundance (measured in RFU) performed on plasma nucleic acids illustrating of intensity and size of cfNA extracted and then subjected to library preparation using a commercial protocol (red) and the methods described herein (black).

FIG. 4 shows yield of canonical and non-canonical cfNA (e.g. cfDNA) using commercial extraction buffers and extraction buffers disclosed herein. Shown is a box plot of each condition showing condition on the x-axis (“commercial” or the methods described herein “Aqtual 4.0”) and various endpoint criteria (% canonical cfDNA fragments of total, canonical cfDNA fragments per mL plasma in nanograms, non-canonical cfDNA fragments per mL plasma in nanograms, or non-canonical to canonical cfDNA yield ratio) on the y-axis.

FIG. 5 shows a genome browser view of regulatory-active chromatin signal on a segment of chromosome 12 representing a promoter region. The first track represents the regulatory region protected from DNase degradation. The subsequent 5 tracks are GM12878 ChIP-Seq −log(p-value) signals for selected histone modifications (H3K4me1, H3K4me2, H3K4me3, H3K27ac, and H3K36me3). The last two bottom tracks represent enrichment of nucleosomal (cfDNA_{nuc}) and regulatory-active chromatin (cfDNA_{ac}) genome-wide signals. Signals were calculated by log2-fold change between the two fragment classes.

FIG. 6 shows a comparison of the average signal distribution surrounding TSS from EPDnew with a +/−10 kb flank. The black arrow shows the gene orientation. The scale of ChIP-Seq distributions is −log(p-value), while the cfDNA_{nuc} and cfDNA_{ac} distributions are scaled coverage.

FIG. 7 shows a comparison of the number of fragments scaled to per million reads within cfDNA_{nuc} and cfDNA_{ac} fragments across different genomic locations. P-values from a 1-sided t-test are shown when cfDNA_{ac} fragments are significantly greater than cfDNA_{nuc} fragments. There is a noticeable enrichment in the number of cfDNA_{ac} fragments across CpG islands (1.6× higher, p<2.2e−308), CpG shores (1.4× higher, p<2.2e−308), and promoters (1.7× higher, p<2.2e−308) when compared to cfDNA_{nuc} fragments.

FIG. 8 shows Pearson correlations between the number of nucleosomal (top) and regulatory chromatin fragments (bottom) isolated as in Example 3 to density of H3K4me1, CTCF, POLR2A, H3K27ac. H3K4me2, H3K4me3, H2A.Z, and EP300 narrow peaks across the genome in 1 Mb segments as assessed from the EPDnew database.

FIG. 9 shows a genome browser view on a segment of chromosome 12 that represent a GM12878 insulator region predicted by GM12878. Top tracks are GM12878 ChIP-Seq −log(p-value) signals for either DNase-treated chromatin (first track) or selected histone modifications (H3K4me1, H3K4me2, H3K4me3, H3K27ac, and H3K36me3). The bottom two tracks represent enrichment of cfDNA_{nuc} and cfDNA_{ac} genome-wide signals. Signals were calculated by log2(fold change) between the two fragment classes.

FIG. 10 shows a comparison of the average signal distribution for different GM12878 chromatin states with a +/−10kb flank for cfDNA_{nuc} (left) and cfDNA_{ac} fragments (right). The y-axis scale for the cfDNA_{nuc} and cfDNA_{ac} distributions represent modified coverage.

FIG. 11a shows cfDNA_{ac} signal (y-axis) of CXCR4 (top), CXCR2 (middle), and CD62L (bottom) across time (x-axis) for sequenced donors. Signal is presented as the mean +/−SEM (standard error of the mean) and p-values correspond to the significance of rhythmicity evaluated by the zero-amplitude test using a period of 24 h.

FIG. 11b shows cfDNA_{ac} signal (y-axis) of CXCR4 (top), CXCR2 (middle), and CD62L (bottom) at different diurnal times (x-axis). p-values correspond to a paired t-test between the diurnal times.

FIG. 12a shows fragment distribution of cfDNA_{nuc} and cfDNA_{ac} across the gene body. Genes from Gencodev42 were stratified into low expressed (grey) and high expressed (red) genes by filtering for the top and bottom 10% expressed genes based on GTEx whole blood median TPM. The genes were scaled to the same length from the transcription start site (TSS) to the transcription end site (TES) with 500 bp unscaled before and after the TES and TSS, respectively.

FIG. 12b shows cfDNA_{ac} signal across the gene body from healthy donor plasma (y-axis) compared to gene expression levels in whole blood defined by GTEx (x-axis). Ten genes with similar expression values were aggregated into “metagenes”.

FIG. 12c shows cfDNA_{ac} signal at promoters defined by Functional Annotation of the Mammalian Genome (FANTOM5) pl sites (y-axis) compared to gene expression levels in whole blood defined by GTEx (x-axis).

FIG. 12d shows cfDNA_{ac} signal across the gene bodies (y-axis) compared to cfDNA_{ac} signal at promoters (x-axis).

FIG. 13 shows sample intensity from an Agilent Cell-Free DNA TapeStation 4200 Assay Electropherogram of cfDNA extracted using Formulation 1 (light gray, n=2) and Formulation 2 (dark gray, n=3).

FIG. 14 shows FANTOM5 TSS locations stratified by GC content, with each bin corresponding to a 20th percentile of the data, and compared the raw fragment counts at these locations. A significant enrichment of cfDNA_{ac} (right) relative to cfDNA_{nuc} (left) were observed regardless of GC content.

FIGS. 15A and 15B show regulatory-active-chromatin fragments correspond to ATAC-Seq and DNAse-I peaks. FIG. 15A shows GM12878 DNase-I narrow peaks from Roadmap Epigenomics and ATAC-Seq narrow peaks from ENCODE were downloaded. FIG. 15B shows ATAC-Seq narrow peaks were lifted over from hg38 to hg19 using UCSC's liftover tool. The number of cfDNA_{ac} (bottom) fragments strongly correlate with both DNase-I (p<2.2e−308) and ATAC-Seq peak densities (p<2.2e−308), while cfDNA_{nuc} (top) did not correlate with either.

FIG. 16 shows the gene body score stratified by the average GC content of the exons, with each bin representing 25th percentile of the data. Regardless of GC content, the spearman correlation with GTEx whole blood TPM was at least 0.93.

FIG. 17 shows the active chromatin workflow (right) resulted in a 3.9- and 5.5-fold increase in the amount of longer, >200 bp and >400 respectively, nucleotides in length cfDNA fragments when compared to the off-the shelf workflows (left).

FIG. 18 shows fragment profiles following cfDNA extraction, library preparation, and sequencing. The post-extraction cfDNA trace shows different distribution from library preparation and sequencing, which is likely attributed to the retention of salts and alcohol, both of which are known to alter migration of DNA fragments. Distribution of DNA fragment lengths from library prep and sequencing show mono-and di-nucleosome cfDNA fragments. Boundaries of nucleosomes (right, shading) used to define cfDNA_{nuc} and cfDNA_{ac} fragments following sequencing.

FIG. 19 depicts a scheme for distinguishing between canonical and non-canonical cfNA fragments without gating directly on sizes. A mixture of short and long cfNA fragments is contacted by two baits which capture distinct cfNA fragments and have a preference toward shorter or longer fragments. For example, bait A may capture shorter fragments and longer fragments and bait B may capture longer fragments (e.g. contain a sequence that only hybridizes to a sequence found in longer fragments). The relative amount of NA fragments hybridized to bait A and bait B is then used to infer the relative abundance of short and long fragments at the targeted genomic region.

FIG. 20 illustrates one example sequencing-based deconvolution where a custom reference collection of sequences of various sizes (e.g. absent absolute or relative genomic position) is mapped to a library of fragments. In this workflow, in some cases the mapped count deconvolution can not involve direct size determination. The relative abundance of short and long fragments derived from one or more genomic regions can be quantified by mapping the sequences of captured fragments to custom references (keywords) as illustrated in FIG. 7. This method does not require determining the absolute length of each captured fragment, mapping fragment sequences to a reference genome, or identifying the ends of individual fragments. Nucleic acids isolated from a biological sample with a mixture of short and long NA fragments are sequenced to determine the nucleotide bases in a representative number of NA fragments. Reference 1 and Reference 2 represent different portions of the genomic site (e.g. a transcriptionally active locus). In the example depicted in FIG. 7, Reference 1 matches both short and long NA fragments, whereas Reference 2 matches only the longer fragments. Each sequenced NA fragment is scored for a match to Reference 1 and Reference 2. An increase in the proportion of NA fragment sequences matching Reference 2 (or Reference 1 and Reference 2) relative to NA fragment sequence that only match Reference 1 indicates an increase in the relative abundance of longer NA fragments at the genomic site.

### DETAILED DESCRIPTION

Disclosed herein are improved methods and compositions for facilitating capture of cfNA (e.g. “non-canonical” cfNA fragments). Such methods can involve particular types of selective anionic capture and solution conditions for favoring enrichment of particular sized cfNA fragments over others.

### Definitions

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4^{th} Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6^{th} Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description or the claims, such terms are intended to be inclusive in a manner similar to the term “comprising”.

The term “about” or “approximately” means within an acceptable error range for the particular value as determined by the application, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

The term “or”, as used herein, is intended to be an inclusive “or”.

The term “nucleic acid” generally refers to deoxyribonucleic acid (DNA), ribonucleic acid (RNA) or any hybrid or fragment thereof. The nucleic acid in the sample can be a cell-free nucleic acid. A sample can be a liquid sample (e.g., blood or interstitial fluid) or a solid sample (e.g., a cell or tissue sample). In some examples, the sample is obtained from a cell-free bodily fluid, such as plasma. In such instance, the sample may include cell-free DNA or cell-free RNA. In some examples, the majority of DNA in a biological sample that may be enriched for cfDNA (e.g., a plasma sample obtained via a centrifugation protocol) can be cell-free (e.g., greater than 50%, 60%, 70%, 80%, 90%, 95%, or 99% of the DNA can be cell-free). In some examples, nucleic acid is derived from circulating tumor cells or circulating fetal cells. Throughout the disclosure the use of cell-free nucleic acid or cfNA may be used interchangeably with nucleic acid or NA.

The term “whole blood sample”, as used herein, generally refers to a whole blood sample that has not been fractionated or separated into its component parts. Whole blood may be combined with an anticoagulant such as ethylenediaminetetraacetic acid (EDTA) or acid citrate dextrose (ACD) during the collection process but is generally otherwise unprocessed. “Whole Blood” may refer to a specific standardized product for transfusion or further processing, or to any unmodified collected blood.

The terms “blood plasma” or “plasma”, as used herein, generally refer to a straw-colored/pale-yellow liquid component of blood that holds the blood cells in whole blood in suspension. Blood plasma can make up about 55% of total blood by volume. It can comprise up to about 93% by volume water, and can contain dissolved proteins including albumins, immunoglobulins, and fibrinogen, glucose, clotting factors, electrolytes (Na+, Ca2+, Mg2+, HCO3 Cl etc.), hormones and carbon dioxide. Blood serum generally refers to blood plasma without fibrinogen or the other clotting factors (i.e., whole blood minus both the cells and the clotting factors).

The term “chaotropic agent” generally refers to a compound that disrupts hydrogen bonds between water molecules and disrupts the tertiary structure of biopolymers. In some embodiments, the chaotropic agent comprises a guanidinium ion (e.g. guanidinium chloride or guanidine thiocyanate), thiourea, or urea. In some embodiments, the chaotropic agent is a sulfur atom-free agent.

As used herein, a “cell” generally refers to a biological cell. A cell may be the basic structural, functional or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an algal cell, an animal cell, a cell from an invertebrate animal, a cell from a vertebrate animal (e.g., fish, amphibian, reptile, bird, mammal), or a cell from a mammal (e.g., a pig, a cow, a goat, a sheep, a rodent, a rat, a mouse, a non-human primate, a human, etc.).

The term “binding agent” generally refers to a molecule that has selective affinity for a biomolecule. Binding agents may comprise aptamers, antibodies, lectins and enzymes.

The term “antibody” generally includes polyclonal antiserum, monoclonal antibodies, fragments of antibodies (e.g. single chain or Fab fragments), and engineered derivatives carrying binding components of antibodies (e.g. single chain variable fragments or ScFvs)

The term “aptamer” generally includes affinity agents with selectivity for a specific predetermined molecule and which are polymers of nucleic acids.

The term “alcohol” generally refers to an organic compound that carries at least one hydroxyl functional group (—OH) bound to a saturated carbon atom. Examples include mono, di, tri, tetra or penta alcohols. Examples further include ethanol or isopropanol. Examples still further include methanol, ethanol, isopropanol, propanol, butanol, ethylene glycol, propylene glycol, butylene glycol, pentylene glycol, glycerol, tetritol, pentitol, 1,3 propane diol, and the like, or mixtures thereof.

The term “chromatin” generally refers to nucleoprotein complexes with nucleic acid which can compact and organize great lengths of cellular genetic material to contain it within cells. The nucleosome, in which double-stranded DNA (dsDNA) is wound approximately twice around a core of conserved histone or histone-like proteins, can comprise a primary level of chromatin organization in the nucleus of eukaryotic cells. Higher-order chromatin organization can involve further compaction of nucleosomes around additional chromatin-associated proteins, and can employ a variety of chromatin assembly factors. The basic structural unit of chromatin is the nucleosome: which can comprise a core octamer of histone proteins (e.g. two copies of each of H2A, H2B, H3 and H4) as well as a linker histone and about 180 base pairs of DNA.

The term “heterochromatin” or “inactive chromatin” generally refers to a subset of chromatin that is most densely compacted and is generally transcriptionally silent.

As used herein, “euchromatin” or “active chromatin” generally refers to more extended chromatin domains that are often transcriptionally active, accessible portions of the genome.

As used herein, the term “H3K4me1” generally refers to monomethylation of lysine 4 in the histone H3 protein. In some embodiments, H3K4mel is associated with active and primed enhancer elements when encountered in a region of cellular chromatin.

As used herein, the term “H3K4me2” generally refers to demethylation of lysine 4 in the histone H3 protein. In some embodiments, H3K4me3 is associated with active transcription of nearby genes when encountered in a region of cellular chromatin.

As used herein, the term “H3K4me3” generally refers to trimethylation of lysine 4 in the histone H3 protein. In some embodiments, H3K4me3 is associated with active transcription of nearby genes when encountered in a region of cellular chromatin.

As used herein, the term “H3K27ac2” or “H3K27ac”generally refers to acetylation of the lysine residue at N-terminal position 27 of the histone H3 protein In some embodiments, H3K27ac is associated with increased transcription when encountered in a region of cellular chromatin.

As used herein, the term “H3K36me3” generally refers to trimethylation at the 36th lysine residue of the histone H3 protein. In some embodiments, H3K36me3 is associated with gene bodies when encountered in a region of cellular chromatin.

As used herein, the term “H2A.Z” generally refers to a variant of histone H2A. In some embodiments, presence of H2A.Z in a region of cellular chromatin can be associated with active chromatin.

### Example embodiments

In some aspects, the present disclosure provides for a method of enriching nucleic acids (NA) comprising active chromatin comprising a sequence of a genomic region. In some aspects, the present disclosure provides for a method of enriching cell-free nucleic acids (cfNA) comprising active chromatin comprising a sequence of a genomic region. In some embodiments, the cfNA comprises cfDNA, cf-mtDNA (mitochondrial DNA), cfRNA, or any combination thereof. Non-limiting examples of cfRNA include miRNA, IncRNA, circRNA, piRNA, YRNA, and vtRNA. In some embodiments, the cfNAs are produced by cell death. In some embodiments, the cfNAs are produced by cleavage of nuclear DNA. In some embodiments, the cfNA produced from cleavage of nuclear DNA is canonical. In some embodiments, the canonical nuclear DNA is about 170 base pairs (bp) in length. In some embodiments, the cfNA produced from cleavage of nuclear DNA is non-canonical. In some embodiments, the non-canonical nuclear DNA greater than 300 bp. In some embodiments, the cfNA originates from tumor cells. In some embodiments, the cfNA originates from lymphoid cells. In some embodiments, the cfNA originates from myeloid cells. In some embodiments, the cfNA originates from fetal cells. In some embodiments, the cfNA originates from microbial cells.

In some embodiments, the cfNA comprises DNA. In some embodiments, the cfNA comprises RNA. In some embodiments, the cfNA is between 10 and 21,000 base pairs. In some embodiments, the cfNA is between 15 and 10,000 base pairs. In some embodiments, the cfNA is canonical. A canonical cfNA fragment may comprise less than 220, 205, 190, or 175 base pairs. A canonical fragment may comprise about 1, 2, 3, 4, 5, 6, 7, 8, 9, 10, 11, 12, 13, 14, 15, 20. 25. 30, 35, 40, 45, 50, 60, 70, 80, 90, 100, 150, 200, 230, or more than 230 base pairs. A canonical fragment may comprise about less than 230, 200, 150, 100, 90, 80, 70, 60, 50, 45, 40, 35, 30, 25, 20, 15, 10, 9, 8, 7, 6, 5, 4, 3, 2, or 1 base pair. A canonical cfNA fragment may comprise less than 220, 205, 190, or 175 base pairs. In some embodiments, the cfNA is non-canonical. A non-canonical cfNA fragment may comprise at least 185, 255, 270, or 310 base pairs. A non-canonical cfNA fragment may comprise about, 200, 250, 300, 350, 400, 450, 500, 600, 700, 800, 900, 1000, or more than 1000 base pairs. A non-canonical cfNA fragment may comprise about 1000, 900, 800, 700, 600, 500, 450, 400, 350, 300, 250, 200, or less than 200 base pairs. In some embodiments the cfNA is between 300 and 1,500 base pairs. In some embodiments, the cfNA may comprise about 170, 200, 250, 300, 350, 400, 450, 500, 600, 700, 800, 900, 1000, 1100, 1200, 1300, 1400, 1500, or more than 1500 base pairs. In some embodiments, the cfNA may comprise about 1500, 1400, 1300, 1200, 1100, 1000, 900, 800, 700, 600, 500, 450, 400, 350, 300, 250, 200, 170, or less than 170 base pairs.

In some embodiments, the cfNA comprises DNA. In some embodiments, the cfNA comprises RNA. In some embodiments, the cfNA is between 10 and 21,000 base pairs in length. In some embodiments, the cfNA is between 15 and 10,000 base pairs in length. In some embodiments, the cfNA is canonical. A canonical cfNA fragment may comprise less than 220, 205, 190, or 175 base pairs in length. A canonical fragment may comprise about 1, 2, 3, 4, 5, 6, 7, 8, 9, 10, 11, 12, 13, 14, 15, 20. 25. 30, 35, 40, 45, 50, 60, 70, 80, 90, 100, 150, 200, 230, or more than 230 base pairs in length. A canonical fragment may comprise about less than 230, 200, 150, 100, 90, 80, 70, 60, 50, 45, 40, 35, 30, 25, 20, 15, 10, 9, 8, 7, 6, 5, 4, 3, 2, or 1 base pair. A canonical cfNA fragment may comprise less than 220, 205, 190, or 175 base pairs in length. In some embodiments, the cfNA is non-canonical. A non-canonical cfNA fragment may comprise at least 185, 255, 270, or 310 base pairs in length. A non-canonical cfNA fragment may comprise about, 200, 250, 300, 350, 400, 450, 500, 600, 700, 800, 900, 1000, or more than 1000 base pairs in length. A non-canonical cfNA fragment may comprise about 1000, 900, 800, 700, 600, 500, 450, 400, 350, 300, 250, 200, or less than 200 base pairs in length. In some embodiments the cfNA is between 300 and 1,500 base pairs in length. In some embodiments, the cfNA may comprise about 170, 200, 250, 300, 350, 400, 450, 500, 600, 700, 800, 900, 1000, 1100, 1200, 1300, 1400, 1500, or more than 1500 base pairs in length. In some embodiments, the cfNA may comprise about 1500, 1400, 1300, 1200, 1100, 1000, 900, 800, 700, 600, 500, 450, 400, 350, 300, 250, 200, 170, or less than 170 base pairs in length.

In some embodiments, the cfNA is between 10 and 21,000 nucleotides. In some embodiments, the cfNA is between 15 and 10,000 nucleotides. In some embodiments, the cfNA is canonical. A canonical cfNA fragment may comprise less than 220, 205, 190, or 175 nucleotides. A canonical fragment may comprise about 1, 2, 3, 4, 5, 6, 7, 8, 9, 10, 11, 12, 13, 14, 15, 20. 25. 30, 35, 40, 45, 50, 60, 70, 80, 90, 100, 150, 200, 230, or more than 230 nucleotides. A canonical fragment may comprise about less than 230, 200, 150, 100, 90, 80, 70, 60, 50, 45, 40, 35, 30, 25, 20, 15, 10, 9, 8, 7, 6, 5, 4, 3, 2, or 1 nucleotides. A canonical cfNA fragment may comprise less than 220, 205, 190, or 175 nucleotides. In some embodiments, the cfNA is non-canonical. A non-canonical cfNA fragment may comprise at least 185, 255, 270, or 310 nucleotides. A non-canonical cfNA fragment may comprise about, 200, 250, 300, 350, 400, 450, 500, 600, 700, 800, 900, 1000, or more than 1000 nucleotides. A non-canonical cfNA fragment may comprise about 1000, 900, 800, 700, 600, 500, 450, 400, 350, 300, 250, 200, or less than 200 nucleotides. In some embodiments the cfNA is between 300 and 1,500 nucleotides. In some embodiments, the cfNA may comprise about 170, 200, 250, 300, 350, 400, 450, 500, 600, 700, 800, 900, 1000, 1100, 1200, 1300, 1400, 1500, or more than 1500 nucleotides. In some embodiments, the cfNA may comprise about 1500, 1400, 1300, 1200, 1100, 1000, 900, 800, 700, 600, 500, 450, 400, 350, 300, 250, 200, 170, or less than 170 nucleotides.

In some embodiments, the cfNA is between 10 and 21,000 nucleotides in length. In some embodiments, the cfNA is between 15 and 10,000 nucleotides in length. In some embodiments, the cfNA is canonical. A canonical cfNA fragment may comprise less than 220, 205, 190, or 175 nucleotides in length. A canonical fragment may comprise about 1, 2, 3, 4, 5, 6, 7, 8, 9, 10, 11, 12, 13, 14, 15, 20. 25. 30, 35, 40, 45, 50, 60, 70, 80, 90, 100, 150, 200, 230, or more than 230 nucleotides in length. A canonical fragment may comprise about less than 230, 200, 150, 100, 90, 80, 70, 60, 50, 45, 40, 35, 30, 25, 20, 15, 10, 9, 8, 7, 6, 5, 4, 3, 2, or 1 nucleotides in length. A canonical cfNA fragment may comprise less than 220, 205, 190, or 175 nucleotides in length. In some embodiments, the cfNA is non-canonical. A non-canonical cfNA fragment may comprise at least 185, 255, 270, or 310 nucleotides in length. A non-canonical cfNA fragment may comprise about, 200, 250, 300, 350, 400, 450, 500, 600, 700, 800, 900, 1000, or more than 1000 nucleotides in length. A non-canonical cfNA fragment may comprise about 1000, 900, 800, 700, 600, 500, 450, 400, 350, 300, 250, 200, or less than 200 nucleotides in length. In some embodiments the cfNA is between 300 and 1,500 nucleotides in length. In some embodiments, the cfNA may comprise about 170, 200, 250, 300, 350, 400, 450, 500, 600, 700, 800, 900, 1000, 1100, 1200, 1300, 1400, 1500, or more than 1500 nucleotides in length. In some embodiments, the cfNA may comprise about 1500, 1400, 1300, 1200, 1100, 1000, 900, 800, 700, 600, 500, 450, 400, 350, 300, 250, 200, 170, or less than 170 nucleotides in length.

In some embodiments, the NA is extracted from blood, urine, stool, interstitial fluid, any subfraction of the aforementioned, or any combination thereof. In some embodiments, the NA is extracted from tissue. In some embodiments, the cfNA is extracted from blood, urine, stool, interstitial fluid, any subfraction of the aforementioned, or any combination thereof. In some embodiments, the NA is extracted from blood. In some embodiments, the NA is extracted from blood plasma. In some embodiments, the cfNA is extracted from blood. In some embodiments, the cfNA is extracted from blood plasma. In some embodiments, the blood is collected by venipuncture, a finger stick, arterial draw, or any combination thereof. In some embodiments, the blood is collected in a clinical setting. Non-limiting examples of clinical settings includes hospital, urgent care, doctor's office, and laboratories. In some embodiments, the blood is collected in a blood collection tube. In some embodiments, the blood collection tube is a STRECK tube. In some cases, the cfNA is extracted from serum or plasma.

In some embodiments, the NA is collected from urine. In some embodiments, the cfNA is collected from urine. In some embodiments, the urine is collected by a subject urinating in a container or through use of a foley catheter. In some embodiments, the container is sterile. In some embodiments, the container is non-sterile. In some embodiments, the container has a lid or seal. The lid may be a cover, a screw top, or pop top. In some embodiments, the seal may be a plug. In some embodiments, the seal may be a zip top. In some embodiments, urine is collected directly from the foley catheter. In some embodiments, the urine is collected from a urinary drainage bag. Urine may be collected in a clinical setting or a personal setting of the subject. Non-limiting examples of clinical settings includes hospital, urgent care, doctor's office, and laboratories. Non-limiting examples of personal settings of the subject may include a house, an office, or a business. In some embodiments, the subject brings the collected urine sample to the clinical setting. In some embodiments, the subject collects the urine by themselves. In some embodiments, the urine is collected by the subject with the help of another person. In some embodiments, the urine is collected by another person. Non-limiting examples of another person includes medical staff such as a nurse, doctor, orderly or a personal contact of the subject such as a family member or friend. In some embodiments, the NA is extracted from stool. In some embodiments, the cfNA is extracted from stool. In some embodiments, the stool is collected by a subject excreting in a container or on to a holder. In some embodiments, the container is sterile. In some embodiments, the container is non-sterile. In some embodiments, the container has a lid or seal. The lid may be a cover, a screw top, or pop top. In some embodiments, the seal may be a plug. In some embodiments, the seal may be a zip top. In some embodiments, the holder is a stick. Stool may be collected in a clinical setting or a personal setting of the subject. Non-limiting examples of clinical settings includes hospital, urgent care, doctor's office, and laboratories. Non-limiting examples of personal settings of the subject may include a house, an office, or a business. In some embodiments, the subject brings the collected stool sample to the clinical setting. In some embodiments, the subject collects the stool by themselves. In some embodiments, the stool is collected by the subject with the help of another person. In some embodiments, the stool is collected by another person. Non-limiting examples of another person includes medical staff such as a nurse, doctor, orderly or a personal contact of the subject such as a family member or friend.

In some embodiments, the NA is extracted from interstitial fluid. In some embodiments, the cfNA is extracted from interstitial fluid. In some embodiments, the interstitial fluid is collected using a needle. In some embodiments, the interstitial fluid is collected in a clinical setting. Non-limiting examples of clinical settings includes hospital, urgent care, doctor's office, and laboratories. In some embodiments, the interstitial fluid is collected in a collection tube. In some embodiments, the interstitial fluid is collected in a container. In some embodiments, the container is sterile. In some embodiments, the container is non-sterile. In some embodiments, the container has a lid or seal. The lid may be a cover, a screw top, or pop top. In some embodiments, the seal may be a plug. In some embodiments, the seal may be a zip top.

In some embodiments, a composition comprising NA in solution is contacted with an anionic solid surface in the presence of a chaotropic agent. In some embodiments, a composition comprising cfNA in solution is contacted with an anionic solid surface in the presence of a chaotropic agent. The anionic solid surface can be any anionic surface. In some embodiments, the anionic solid surface is planar or spherical. In some embodiments, the anionic solid surface is a slide, a flow cell, a well, a bead, or any combination thereof.

An anionic solid surface may be a planar surface. A planar surface may be the interior of a well. A well may have a dimension of x by y by z, where x, y, and z are each independently at least about 0.1 μm, 1 μm, 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 250 μm, 300 μm, 400 μm, 500 μm, 600 μm, 700 μm, 800 μm, 900 μm, 1,000 μm, or more micrometers. A well may have a dimension of x by y by z, where x, y, and z are each independently at most about 1,000 μm, 900 μm, 800 μm, 700 μm, 600 μm, 500 μm, 400 μm, 300 μm, 250 μm, 200 μm, 190 μm, 180 μm, 170 μm, 160 μm, 150 μm, 140 μm, 130 μm, 120 μm, 110 μm, 100 μm, 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 10 μm, 5 μm, 1 μm, 0.1 μm, or less micrometers. For example, a well can have an x dimension of 434 μm, a y dimension of 30 μm, and a z dimension of 510 μm. In another example, a well can have an x and y dimension of 16 μm and a z dimension of 1 μm. The planar surface may be a well among a plurality of wells. The plurality of wells may comprise at least two wells. The plurality of wells may comprise at least 1,000 wells. There may be 2, 3, 4, 5, 6, 7, 8, 9, 10, 15, 20, 25, 30, 35, 40, 45, 50, 60, 70, 80, 90, 100, 200, 300, 400, 500, 1000, 1500, 2000, 3000, 4000, 5000, 10,000, 100,000,1,000,000 or more than 1,000,000 wells in a plurality of wells. A well may comprise a bead or a planar surface may incorporate a bead.

An anionic solid surface may be a bead or nanoparticle. A bead may be a polymer such as a polystyrene bead or polystyrene cross-linked with divinylbenzene. In some embodiments, the bead comprises a single composition. A bead may be cellulose, cellulose derivatives, gelatin, acrylic resins, glass, silica gels, polyvinyl pyrrolidine (PVP), co-polymers of vinyl and acrylamide, polyacrylamides, latex gels, dextran, crosslinked dextran (e.g., Sephadex™), rubber, silicon, plastics, nitrocellulose, natural sponges, metal, and agarose gel (Sepharose™). Non-limiting examples of single compositions include silicon dioxide (e.g., glass), metal, and plastic. In some embodiments, the bead comprises multiple components. In some embodiments, the bead comprises a bead core and an outer shell.

In some embodiments, the bead core comprises metal. In some embodiments, the metal bead core is magnetic. In some embodiments, the magnetic metal bead core comprises metal oxide. In some embodiments, the metal oxide is iron oxide. In some embodiments, the iron oxide is iron (II, III) oxide. In some embodiments, the iron oxide is black iron oxide. In some embodiments, the iron oxide molecule further comprises an additional metal element. In some embodiments, the metal element is an alkaline earth metal. Non limiting examples of alkaline earth metals includes magnesium, calcium, and barium. In some embodiments, the metal element is a transition metal. Non-limiting examples of transition metals includes manganese, cobalt, copper, zinc, nickel, and chromium. A bead may comprise a metal salt such as a copper salt, a magnesium salt, a calcium salt, or a manganese salt.

In some embodiments, an outer shell covers the bead core. In some embodiments, the outer shell of the bead is the anionic solid surface. In some embodiments, the outer shell comprises polymer, such as polyethylene glycol, polyacrylic acid, polyacrylamide, polyvinyl alcohol, poly-methyl methacrylate, polystyrene, poly-4-vinylphenol, polyester, polyimide, polyethylene, polypropylene, polyethylene vinyl acetate, polyacrylates, polysaccharide, etc. In some embodiments, the polymer has different molecular weight. In various embodiments, the polymer has an average molecular weight of 100 to 500000 Dalton, such as 200 to 100000 Dalton, 300 to 50000 Dalton, 400 to 20000 Dalton, 500 to 10000 Dalton, or 600 to 5000 Dalton. In some embodiments, the polymer comprises one monomer unit. In some of these embodiments, the monomer is ethylene glycol, acrylic acid, acrylamide, or styrene. In some embodiments, the polymer is a copolymer comprising two or more different monomer units. In some of these embodiments, the two or more different monomer units are selected from ethylene glycol, acrylic acid, acrylamide, and styrene. In some embodiments, the polymer has a linear structure. In some embodiments, the polymer has a branched structure. In some embodiments, the polymer is cross-linked.

In some embodiments, the outer shell has a thickness of less than 300 nm, such as less than 250 nm, less than 200 nm, less than 150 nm, less than 100 nm, less than 80 nm, less than 50 nm, less than 20 nm, less than 10 nm, less than 5 nm, less than 2 nm, or less than 1 nm. In various embodiments, the outer shell has a thickness of 1 nm to 300 nm, such as 1 nm to 250 nm, 1 nm to 200 nm, 1 nm to 150 nm, 1 nm to 100 nm, 1 nm to 50 nm, 1 nm to 10 nm, 10 nm to 300 nm, 10 nm to 250 nm, 10 nm to 200 nm, 10 nm to 150 nm, 10 nm to 100 nm, 10 nm to 50 nm, 50 nm to 300 nm, 50 nm to 250 nm, 50 nm to 200 nm, 50 nm to 150 nm, 50 nm to 100 nm, 100 nm to 300 nm, 100 nm to 250 nm, 100 nm to 200 nm, 100 nm to 150 nm, 150 nm to 300 nm, 150 nm to 250 nm, 150 nm to 200 nm, 200 nm to 300 nm, 200 nm to 250 nm, or 250 nm to 300 nm. In certain embodiments, the outer shell has a thickness of about 250 nm. In certain embodiments, the outer shell has a thickness of about 200 nm. In certain embodiments, the outer shell has a thickness of about 150 nm. In certain embodiments, the outer shell has a thickness of about 100 nm. In certain embodiments, the outer shell has a thickness of about 80 nm. In certain embodiments, the outer shell has a thickness of about 60 nm. In certain embodiments, the outer shell has a thickness of about 40 nm. In certain embodiments, the outer shell has a thickness of about 20 nm. In certain embodiments, the outer shell has a thickness of about 10 nm. In certain embodiments, the outer shell has a thickness of about 5 nm. In certain embodiments, the outer shell has a thickness of about 2 nm. In certain embodiments, the outer shell has a thickness of about 1 nm.

In some embodiments, the shell is a single layer. In other embodiments, the shell comprises a plurality of layers. In certain embodiments, the shell comprises a layer of silicon dioxide and a layer of titanium dioxide. In certain embodiments, the shell comprises a layer of silicon dioxide and a layer of polymer. In certain embodiments, the shell comprises a layer of titanium dioxide and a layer of polymer.

In some embodiments, the shell comprises at least one nonporous layer. In certain embodiments, the shell comprises a nonporous silicon dioxide layer. In certain embodiments, the shell comprises a nonporous titanium dioxide layer. In certain embodiments, the shell comprises a nonporous polymer layer. In certain embodiments, the shell comprises a layer of nonporous silicon dioxide and a layer of nonporous titanium dioxide. In certain embodiments, the shell comprises a layer of nonporous silicon dioxide and a layer of nonporous polymer. In certain embodiments, the shell comprises a layer of nonporous titanium dioxide and a layer of nonporous polymer.

In certain embodiments, the outer shell comprises at least one layer with mesoporous structure. In certain embodiments, the outer shell comprises a layer of mesoporous silicon dioxide. In certain embodiments, the outer shell comprises a layer of mesoporous titanium dioxide. In certain embodiments, the outer shell comprises a layer of mesoporous polymer. In some embodiments, the outer shell comprises a layer of nonporous material and a layer of mesoporous material. In some of these embodiments, the layer of mesoporous material covers the layer of nonporous material. In some embodiments, the outer shell comprises a layer of nonporous silicon dioxide and a layer of mesoporous silicon dioxide. In some of these embodiments, the layer of mesoporous silicon dioxide covers the layer of nonporous silicon dioxide. In some embodiments, the outer shell comprises a layer of nonporous titanium dioxide and a layer of mesoporous titanium dioxide. In some of these embodiments, the layer of mesoporous titanium dioxide covers the layer of nonporous titanium dioxide. In some embodiments, the outer shell comprises a layer of nonporous polymer and a layer of mesoporous polymer. In some of these embodiments, the layer of mesoporous polymer covers the layer of nonporous polymer. In some embodiments, the outer shell comprises a layer of nonporous silicon dioxide and a layer of mesoporous titanium dioxide. In some of these embodiments, the layer of mesoporous titanium dioxide covers the layer of nonporous silicon dioxide. In some embodiments, the outer shell comprises a layer of nonporous titanium dioxide and a layer of mesoporous silicon dioxide. In some of these embodiments, the layer of mesoporous silicon dioxide covers the layer of nonporous titanium dioxide. In some embodiments, the outer shell comprises a layer of nonporous polymer and a layer of mesoporous silicon dioxide. In some of these embodiments, the layer of mesoporous silicon dioxide covers the layer of nonporous polymer. In some embodiments, the outer shell comprises a layer of nonporous silicon dioxide and a layer of mesoporous polymer. In some of these embodiments, the layer of mesoporous polymer covers the layer of nonporous silicon dioxide. In some embodiments, the outer shell comprises a layer of nonporous polymer and a layer of mesoporous titanium dioxide. In some of these embodiments, the layer of mesoporous titanium dioxide covers the layer of nonporous polymer. In some embodiments, the outer shell comprises a layer of nonporous titanium dioxide and a layer of mesoporous polymer. In some of these embodiments, the layer of mesoporous polymer covers the layer of nonporous titanium dioxide.

In some embodiments, the outer shell contains a coating. In some embodiments, the outer shell contains functional groups. In some embodiments, the functional group is attached to the outer shell. In some embodiments, the functional group is covalently attached to the outer shell. In some embodiments, the functional group is attached to the outer shell non-covalently. In some embodiments, the functional group is capable of binding directly to a predetermined molecule, such as a nucleic acid, a protein, a peptide, a carbohydrate, a lipid, or an organic molecule. In certain embodiments, the functional group is capable of binding to a molecular probe, such as a nucleic acid probe or a protein probe. In some of these latter embodiments, the molecular probe is capable, in turn, of binding to a predetermined molecule, such as a nucleic acid, a protein, a peptide, a carbohydrate, or a lipid. In various embodiments, the functional group is carboxyl, hydroxyl, epoxy, carbonyl, aldehyde, amine, maleimide, N-hydroxysuccinimide, carbodiimide, anhydride, hydrazide, polyethylene glycol, azide, nitrile, sulfhydryl, thiocyanate, phosphate, borono, thioester, cysteine, disulfide, alkyl and acyl halide, glutathione, maltose, isocyanate, sulfonyl chloride, tosylate ester, carbonate, arylating agent, imidoester, fluorophenyl ester, or Schiff base. In some embodiments, the functional group can be carboxyl, hydroxyl, epoxy, carbonyl, aldehyde, amine, maleimide, N-hydroxysuccinimide, carbodiimide, anhydride, hydrazide, or biotin. In some embodiments, the magnetic particle comprises a plurality of functional group species. In some embodiments, each of the plurality of functional groups is attached to the outer shell. In various embodiments, the functional group lead to the creation of surface charge of the magnetic nanoparticle. In some embodiments, the surface charge of the magnetic nanoparticle is positive. In some embodiments, the surface charge of the magnetic nanoparticle is negative. In some embodiments, the surface charge of the magnetic nanoparticle can be tuned by changing the pH of the solution.

In some embodiments, the bead has a maximum diameter of less than 1 μm, such as less than 900 nm, less than 800 nm, less than 700 nm, less than 600 nm, less than 500 nm, less than 400 nm, less than 300 nm, or less than 200 nm. In various embodiments, the magnetic nanoparticle has a maximum diameter of 100 nm to 1000 nm, such as 100 nm to 900 nm, 100 nm to 800 nm, 100 nm to 700 nm, 100 nm to 600 nm, 100 nm to 500 nm, 100 nm to 400 nm, 100 nm to 300 nm, 100 nm to 200 nm, 200 nm to 1000 nm, 200 nm to 900 nm, 200 nm to 800 nm, 200 nm to 700 nm, 200 nm to 600 nm, 200 nm to 500 nm, 200 nm to 400 nm, 200 nm to 300 nm, 300 nm to 1000 nm, 300 nm to 900 nm, 300 nm to 800 nm, 300 nm to 700 nm, 300 nm to 600 nm, 300 nm to 500 nm, 300 nm to 400 nm, 400 nm to 1000 nm, 400 nm to 900 nm, 400 nm to 800 nm, 400 nm to 700 nm, 400 nm to 600 nm, 400 nm to 500 nm, 500 nm to 1000 nm, 500 nm to 900 nm, 500 nm to 800 nm, 500 nm to 700 nm, 500 nm to 600 nm, 600 nm to 1000 nm, 600 nm to 900 nm, 600 nm to 800 nm, 600 nm to 700 nm, 700 nm to 1000 nm, 700 nm to 900 nm, 700 nm to 800 nm, 800 nm to 1000 nm, 800 nm to 900 nm, or 900 nm to 1000 nm. In certain embodiments, the magnetic core has a maximum diameter of about 900 nm. In certain embodiments, the magnetic core has a maximum diameter of about 800 nm. In certain embodiments, the magnetic core has a maximum diameter of about 700nm. In certain embodiments, the magnetic core has a maximum diameter of about 600 nm. In certain embodiments, the magnetic core has a maximum diameter of about 500 nm. In certain embodiments, the magnetic core has a maximum diameter of about 400 nm. In certain embodiments, the magnetic core has a maximum diameter of about 300 nm. In certain embodiments, the magnetic core has a maximum diameter of about 200 nm. The bead diameter may depend on the sample assayed requiring smaller or larger beads. The solid support may be of a specific size diameter to allow for the selective binding of non-canonical fragments. The solid support may be modified with various functional groups such as carboxyl, hydroxyl, amino, etc. to promote nucleic acid isolation.

In some embodiments, the magnetic core has a maximum diameter of less than 1 μm, such as less than 800 nm, less than 700 nm, less than 600 nm, less than 500 nm, less than 400 nm, less than 300 nm, or less than 200 nm. In various embodiments, the magnetic core has a maximum diameter of 100 nm to 800 nm, such as 100 nm to 700 nm, 100 nm to 600 nm, 100 nm to 500 nm, 100 nm to 400 nm, 100 nm to 300 nm, 100 nm to 200 nm, 200 nm to 800 nm, 200 nm to 700 nm, 200 nm to 600 nm, 200 nm to 500 nm, 200 nm to 400 nm, 200 nm to 300 nm, 300 nm to 800 nm, 300 nm to 700 nm, 300 nm to 600 nm, 300 nm to 500 nm, 300 nm to 400 nm, 400 nm to 800 nm, 400 nm to 700 nm, 400 nm to 600 nm, 400 nm to 500 nm, 500 nm to 800 nm, 500 nm to 700 nm, 500 nm to 600 nm, 600 nm to 800 nm, 600 nm to 700 nm, or 700 nm to 800 nm. In certain embodiments, the magnetic core has a maximum diameter of about 700 nm. In certain embodiments, the magnetic core has a maximum diameter of about 600 nm. In certain embodiments, the magnetic core has a maximum diameter of about 500 nm. In certain embodiments, the magnetic core has a maximum diameter of about 450 nm. In certain embodiments, the magnetic core has a maximum diameter of about 400 nm. In certain embodiments, the magnetic core has a maximum diameter of about 350 nm. In certain embodiments, the magnetic core has a maximum diameter of about 300 nm. In certain embodiments, the magnetic core has a maximum diameter of about 250 nm. In certain embodiments, the magnetic core has a maximum diameter of about 200 nm.

A bead may be a single bead or may be among a plurality of beads. The plurality of beads may comprise at least 1,000 wells. There may be 2, 3, 4, 5, 6, 7, 8, 9, 10, 15, 20, 25, 30, 35, 40, 45, 50, 60, 70, 80, 90, 100, 200, 300, 400, 500, 1000, 1500, 2000, 3000, 4000, 5000, 10,000, 100,000, 1,000,000 or more than 1,000,000 wells in a plurality of beads.

In some embodiments, the extraction of NAs utilizes a lysis buffer. In some embodiments, the extraction of cfNAs utilizes a lysis buffer. In some embodiments, the lysis buffer comprises a chaotropic agent. The chaotropic agent can be any chaotropic agent. Non-limiting examples of chaotropic agents include guanidinium-based solutions (e.g., guanidinium hydrochloride, guanidinium chloride, guanidinium thiocyanate, guanidinium isothiocyanate), urea-based solutions (e.g., urea, thiourea), lithium-based solutions (e.g., lithium perchlorate, lithium acetate), propylene glycol, phenol, and DMSO. In some embodiments, the chaotropic agent is a salt. In some embodiments, the chaotropic agent is a guanidinium salt. In some embodiments, the guanidinium salt is guanidinium isothiocyanate. In some embodiments, the final concentration of the chaotropic agent is about 0.40 M to about 0.20 M. In some embodiments, the final concentration of the chaotropic agent is about 0.35 M to about 0.25 M. In some embodiments, the final concentration of the chaotropic agent is about 0.30 M to about 0.25 M. In some embodiments, the final concentration of the chaotropic agent is about 0.25 M. In some embodiments, the final concentration of the chaotropic agent is about 0.26 M. In some embodiments, the lysis buffer contains an enzyme. In some embodiments the enzyme is a protease (e.g., proteolytic enzyme). In some embodiments, the protease is proteinase K. In some embodiments, the final amount of proteinase K used in a reaction is no more than about 3%. In some embodiments, the final amount of proteinase K used in a reaction is no less than about 1%. In some embodiments, the final amount of proteinase K used in a reaction is about 3% to about 1%. In some embodiments, the final amount of proteinase K used in a reaction is about 2% to about 1%. In some embodiments, the final amount of proteinase K used in a reaction is about 1.75% to about 1.25%. In some embodiments, the final amount of proteinase K used in a reaction is about 1.75% to about 1.50%. In some embodiments, the final amount of proteinase K used in a reaction is about 1.7% to about 1.6%. In some embodiments, the lysis buffer has a pH from about 6 to about 9. In some embodiments, the lysis buffer has a pH no more than 9. In some embodiments, the lysis buffer has a pH no less than 6. In some embodiments, lysis buffer has a pH of about 8. In some embodiments, the lysis buffer has a pH of 6.5 or less or 6.0 or less. In some embodiments, lysis buffer has a pH of 8. In some embodiments, the lysis buffer has a pH of between 6.0 and 8.5, between 6.0 and 8.0, between 6.0 and 7.5, between 6.0 and 7.0, or between 6.0 and 6.5. In some embodiments, the lysis buffer incubates with the sample at a temperature higher than room temperature. In some embodiments, the lysis buffer incubates with the sample at a temperature no higher than 70° C. In some embodiments, the lysis buffer incubates with the sample at a temperature no lower than 50° C. In some embodiments, the lysis buffer incubates with the sample at a temperature from about 70° C. to about 50° C. In some embodiments, the lysis buffer incubates with the sample at a temperature from about 65° C. to about 55° C. In some embodiments, the lysis buffer incubates with the sample at a temperature from about 60° C. to about 55° C. In some embodiments, the lysis buffer incubates with the sample at a temperature of about 58° C. In some embodiments, the lysis buffer incubates with the sample for no more than 60 minutes. In some embodiments, the lysis buffer incubates with the sample for no less than 15 minutes. In some embodiments, the lysis buffer incubates with the sample for about 60 minutes to about 15 minutes. In some embodiments, the lysis buffer incubates with the sample for about 40 minutes to about 20 minutes. In some embodiments, the lysis buffer incubates with the sample for about 35 minutes to about 25 minutes. In some embodiments, the lysis buffer incubates with the sample for about 30 minutes.

In some embodiments, the extraction of NAs utilizes a binding buffer. In some embodiments, the extraction of cfNAs utilizes a binding buffer. In some embodiments, the binding buffer comprises a chaotropic agent. The chaotropic agent can be any chaotropic agent. Non-limiting examples of chaotropic agents include guanidinium-based solutions (e.g., guanidinium hydrochloride, guanidinium chloride, guanidinium thiocyanate, guanidinium isothiocyanate), urea-based solutions (e.g., urea, thiourea), lithium-based solutions (e.g., lithium perchlorate, lithium acetate), propylene glycol, phenol, and DMSO. In some embodiments, the chaotropic agent is a salt. In some embodiments, the chaotropic agent is a guanidinium salt. In some embodiments, the guanidinium salt is guanidinium isothiocyanate. In some embodiments, the final concentration of the chaotropic agent is about 2.5 M to about 1.5 M. In some embodiments, the final concentration of the chaotropic agent is about 2 M to about 1.5 M. In some embodiments, the final concentration of the chaotropic agent is about 1.8 M. In some embodiments, the binding buffer has a pH of 6.5 or less or 6.0 or less. In some embodiments, lysis buffer has a pH of 8. In some embodiments, the lysis buffer has a pH of between 6.0 and 8.5, between 6.0 and 8.0, between 6.0 and 7.5, between 6.0 and 7.0, or between 6.0 and 6.5. In some embodiments, the binding buffer comprises an alcohol. The alcohol can be any alcohol, such as ethyl alcohol or isopropyl alcohol. In some embodiments, the alcohol is isopropyl alcohol. In some embodiments, the final concentration of alcohol mixed with the sample is no more than about 15%, 14%, 13%, 12%, 11%, 10%, 9.5%, 9%, 8.5%, 8%, 7.5%, 7%, 6.5%, 6%, 5.5%, 5%, 4.5%, 4%, 3.5%, 3%, 2.5%, 2%, 1.5% or 1%, or any range between these values. In some embodiments, the final concentration of alcohol mixed with the sample is no less than about 2%, 2.5%, 3%, or 3.5%. In some embodiments, the final concentration of alcohol mixed with the sample is about 15% to about 2%. In some embodiments, the final concentration of alcohol mixed with the sample is about 11% to about 3.5%. In some embodiments, the final concentration of alcohol mixed with the sample is about 9% to about 6%. In some embodiments, the final concentration of alcohol mixed with the sample is about 8% to about 7%. In some embodiments, the final concentration of alcohol mixed with the sample is about 7.5% to about 7%. In some embodiments, the final concentration of alcohol mixed with the sample is about 7.25%. In some embodiments, the final concentration of alcohol mixed with the sample is about 7.3%. In some embodiments, the binding buffer comprises a detergent. The detergent can be any suitable detergent used to solubilize biological samples. In some embodiments, the detergent is an ionic detergent (e.g. an anionic or cationic detergent). Non-limiting examples of ionic detergents include dodecylsulfide salts (e.g. sodium dodecylsulfide or SDS), bile acid salts (e.g. Sodium cholate and sodium deoxycholate), and quaternary ammonium detergents (e.g. cetyltrimethylammonium bromide or CTAB). In some embodiments, the detergent is a nonionic detergent. Non-limiting examples of classes of nonionic detergents include hydrophilic polyethylene oxide derivatives (e.g. Triton™ X-100, Triton™ X-102, Triton™ X-114, Triton™ CG-110, Triton™ X-405, Triton™ X-165, Triton™ X-45, Triton™ N-57, Triton™ N-60), lipid-like nonionic detergents (e.g. n-Dodecyl-beta-Maltoside or DDM), steroidal nonionic detergents (e.g. digitonin), nonionic polyoxyethylene detergents (e.g. Tween®-20, Tween®40, Tween®-60, Tween®-80, Tween®65, Tween®-85, Tagat TO, Cremophore RH 40, Cremophore EL, Alpha-tocopherol TGPS, Brij®-96, Brij®-S20, Brij®-S100, Brij®-35, Brij®-58, Brij®-020, Brij®-L23, Brij®-S10, Brij®-010, Brij®-C10, Brij®-93, Brij®-L4, SP Brij® C2 MBAL-SO-(SG), and SP Brij® S2 MBAL), and nonionic ethoxylated nonylphenol detergents (e.g. NP-40). In some embodiments the nonionic detergent is a hydrophilic polyethylene oxide derivative. In some embodiments, the nonionic detergent is a lipid-like nonionic detergent. In some embodiments, the nonionic detergent is a steroidal nonionic detergent. In some embodiments, the nonionic detergent is a polysorbate-type nonionic detergent. In some embodiments, the nonionic detergent is a nonionic polyoxyethylene detergent. In some embodiments, the nonionic detergent is a ethoxylated nonylphenol detergent (e.g. NP-40). In some embodiments, the final concentration of detergent mixed with the sample is no more than about 6%. In some embodiments, the final concentration of detergent mixed with the sample is no less than about 2%. In some embodiments, the final concentration of detergent mixed with the sample is about 6% to about 2%. In some embodiments, the final concentration of detergent mixed with the sample is about 5% to about 3%. In some embodiments, the final concentration of detergent mixed with the sample is about 4% to about 3%. In some embodiments, the final concentration of detergent mixed with the sample is about 3.5% to about 3%. In some embodiments, the final concentration of detergent mixed with the sample is about 3.25%. In some embodiments, the final concentration of detergent mixed with the sample is about 3.3%. In some embodiments, the binding buffer has a pH from about 6 to about 9. In some embodiments, the binding buffer has a pH from about 6 to about 8.5. In some embodiments, the binding buffer has a pH of about 8. In some embodiments, the binding buffer has a pH of 7.8. In some embodiments, the binding buffer incubates with the sample at a temperature no higher than about 25° C. In some embodiments, the binding buffer incubates with the sample at a temperature no lower than 15° C. In some embodiments, the binding buffer incubates with the sample at a temperature from about 25° C. to about 15° C. In some embodiments, the binding buffer incubates with the sample at a temperature from about 22° C. to about 18° C. In some embodiments, the binding buffer incubates with the sample at a temperature of about 20° C. In some embodiments, the binding buffer incubates with the sample at room temperature. In some embodiments, the binding buffer incubates with the sample for no more than 60 minutes. In some embodiments, the binding buffer incubates with the sample for no less than 15 minutes. In some embodiments, the binding buffer incubates with the sample for about 60 minutes to about 15 minutes. In some embodiments, the binding buffer incubates with the sample for about 40 minutes to about 20 minutes. In some embodiments, the binding buffer incubates with the sample for about 35 minutes to about 25 minutes. In some embodiments, the binding buffer incubates with the sample for about 30 minutes. In some embodiments, the binding buffer incubates with the sample undisturbed. In some embodiments, the binding buffer incubates with the sample with disruption. In some embodiments, the disruption is shaking. In some embodiments, the disruption is on a tube rotator.

In some embodiments, NA not bound to the anionic solid surface is removed. In some embodiments, cfNA not bound to the anionic solid surface is removed. In some embodiments, the unbound NA is removed with a wash buffer comprising a wash operation. In some embodiments, the unbound cfNA is removed with a wash buffer comprising a wash operation. In some embodiments, more than one wash operation is performed. In some embodiments, more than one wash buffer is used. In some embodiments, the extraction of NAs utilizes a wash buffer. In some embodiments, the extraction of cfNAs utilizes a wash buffer. In some embodiments, the extraction of cfNAs utilizes more than 1 wash buffer. In some embodiments, the extraction of cfNAs utilizes two different wash buffers. In some embodiments, the first wash buffer comprises a chaotropic agent. The chaotropic agent can be any chaotropic agent. Non-limiting examples of chaotropic agents include guanidinium-based solutions (e.g., guanidinium hydrochloride, guanidinium chloride, guanidinium thiocyanate, guanidinium isothiocyanate), urea-based solutions (e.g., urea, thiourea), lithium-based solutions (e.g., lithium perchlorate, lithium acetate), propylene glycol, phenol, and DMSO. In some embodiments, the chaotropic agent is a salt. In some embodiments, the chaotropic agent is a guanidinium salt. In some embodiments, the guanidinium salt is guanidinium isothiocyanate. In some embodiments, the final concentration of the chaotropic agent is about 4M (molar), about 3.75M, about 3.5M, about 3.25M, about 3.0 M, about 2.75M, about 2.5M, about 2.0M, about 1.75M, about 1.5M, about 1.25M, about 1 M, about 0.75M, about 0.5 M, or about 0.25M, or any range between these values. In some embodiments, the final concentration of the chaotropic agent is about 3.5 M to about 1.5 M. In some embodiments, the final concentration of the chaotropic agent is about 3 M to about 2 M. In some embodiments, the final concentration of the chaotropic agent is about 2.5 M. In some embodiments, the first wash buffer comprises an alcohol. The alcohol can be any alcohol, such as ethyl alcohol or isopropyl alcohol. In some embodiments, the alcohol is ethyl alcohol. In some embodiments, the final concentration of alcohol mixed with the sample is no more than about 30%, 25%, 20%, 15%, 10%, 5%, or 1%, or any range between these values. In some embodiments, the final concentration of alcohol mixed with the sample is no less than about 5%. In some embodiments, the final concentration of alcohol mixed with the sample is about 30% to about 5%. In some embodiments, the final concentration of alcohol mixed with the sample is about 25% to about 5%. In some embodiments, the final concentration of alcohol mixed with the sample is about 20% to about 10%. In some embodiments, the final concentration of alcohol mixed with the sample is about 16% to about 14%. In some embodiments, the final concentration of alcohol mixed with the sample is about 15%. In some embodiments, the first wash buffer comprises a detergent. The detergent can be any suitable detergent used to solubilize biological samples. In some embodiments, the detergent is an ionic detergent (e.g. an anionic or cationic detergent). Non-limiting examples of ionic detergents include dodecylsulfide salts (e.g. sodium dodecylsulfide or SDS), bile acid salts (e.g. Sodium cholate and sodium deoxycholate), and quaternary ammonium detergents (e.g. cetyltrimethylammonium bromide or CTAB). In some embodiments, the detergent is a nonionic detergent. Non-limiting examples of classes of nonionic detergents include hydrophilic polyethylene oxide derivatives (e.g. Triton™ X-100, Triton™ X-102, Triton™ X-114, Triton™ CG-110, Triton™ X-405, Triton™ X-165, Triton™ X-45, Triton™ N-57, Triton™ N-60), lipid-like nonionic detergents (e.g. n-Dodecyl-beta-Maltoside or DDM), steroidal nonionic detergents (e.g. digitonin), nonionic polyoxyethylene detergents (e.g. Tween®-20, Tween®40, Tween®-60, Tween®-80, Tween@65, Tween®-85, Tagat TO, Cremophore RH 40, Cremophore EL, Alpha-tocopherol TGPS, Brij®-96, Brij®-S20, Brij®-S100, Brij®-35, Brij®-58, Brij®-020, Brij®-L23, Brij®-S10, Brij®-010, Brij®-C10, Brij®-93, Brij®-L4, SP Brij® C2 MBAL-SO-(SG), and SP Brij® S2 MBAL), and nonionic ethoxylated nonylphenol detergents (e.g. NP-40). In some embodiments the nonionic detergent is a hydrophilic polyethylene oxide derivative. In some embodiments, the nonionic detergent is a lipid-like nonionic detergent. In some embodiments, the nonionic detergent is a steroidal nonionic detergent. In some embodiments, the nonionic detergent is a polysorbate-type nonionic detergent. In some embodiments, the nonionic detergent is a nonionic polyoxyethylene detergent. In some embodiments, the nonionic detergent is a ethoxylated nonylphenol detergent (e.g. NP-40). In some embodiments, the final concentration of detergent mixed with the sample is no more than about 5%, 4.5%, 4%, 3.5%, 3%, 2.5%, 2%, 1.5%, 1%, or 0.5%, or any range between these values. In some embodiments, the final concentration of detergent mixed with the sample is no less than about 1%. In some embodiments, the final concentration of detergent mixed with the sample is about 5% to about 1%. In some embodiments, the final concentration of detergent mixed with the sample is about 4% to about 2%. In some embodiments, the final concentration of detergent mixed with the sample is about 3.5% to about 2.5%. In some embodiments, the final concentration of detergent mixed with the sample is about 3%. In some embodiments, the first wash buffer has a pH from about 6 to about 9. In some embodiments, the first wash buffer has a pH from about 6 to about 8.5. In some embodiments, the first wash buffer has a pH of about 8, 7.75, 7.5. 7.25, 7.0, 6.75, 6.5, or 6, or any range between these values. In some embodiments, the first wash buffer has a pH of 8.1. In some embodiments, the first wash buffer has a pH of greater than or equal to about 6.5 or 6.0. In some embodiments, the first wash buffer is used to wash the sample once. In some embodiments, the second wash buffer comprises an alcohol. The alcohol can be any alcohol, such as ethyl alcohol or isopropyl alcohol. In some embodiments, the alcohol is ethyl alcohol. In some embodiments, the final concentration of alcohol mixed with the sample is no more than about 90%, 88%, 86%, 84%, 82%, 80%, 78%, 76%, 74%, 72%, or 70%, or any range between these values. In some embodiments, the final concentration of alcohol mixed with the sample is no less than about 70%. In some embodiments, the final concentration of alcohol mixed with the sample is about 90% to about 70%. In some embodiments, the final concentration of alcohol mixed with the sample is about 85% to about 75%. In some embodiments, the final concentration of alcohol mixed with the sample is about 80%. In some embodiments, the second wash buffer is used to wash the sample at least once. In some embodiments, the second wash buffer is used to wash the sample twice. In some embodiments, the second wash buffer has a pH from about 6 to about 9. In some embodiments, the second wash buffer has a pH of 6.0, 6.25, 6.5, 6.75, 7.0, 7.25, 7.5, 8.0, 8.25, 8.5, 8.75, or 9.0, or any range between these values.

In some embodiments, the second wash buffer has a pH from about 6 to about 8.5. In some embodiments, the second wash buffer has a pH of about 8.

In some embodiments, the NA bound the anionic solid surface are eluted. In some embodiments, the cfNA bound the anionic solid surface are eluted. In some embodiments, the NA bound is eluted with an elution operation. In some embodiments, the cfNA bound is eluted with an elution operation. In some embodiments, the elution operation utilizes an elution buffer. In some embodiments, the elution solution is basic. In some embodiments, the extraction of cfNAs utilizes an elution buffer. In some embodiments, the elution buffer comprises tris-base buffer. In some embodiments, the elution buffer has a pH from about 6 to about 9. In some embodiments, the elution buffer has a pH of 6.0, 6.25, 6.5, 6.75, 7.0, 7.25, 7.5, 8.0, 8.25, 8.5, 8.75, or 9.0, or any range between these values. In some embodiments, the elution buffer has a pH from about 6.5 to about 9. In some embodiments, the elution buffer has a pH of about 9. In some embodiments, the elution buffer has a pH of 9.

In some embodiments, the buffers used for NA extraction have a pH that is basic. In some embodiments, the buffers used for NA extraction have a pH that is slightly acidic. In some embodiments, the buffers used for NA extraction have a pH that is neutral. In some embodiments, the pH range of the buffers used for NA extraction have a pH from about 6 to about 9. In some embodiments, the pH of the buffers used for NA extraction is 6.0, 6.25, 6.5, 6.75, 7.0, 7.25, 7.5, 8.0, 8.25, 8.5, 8.75, or 9.0, or any range between these values. In some embodiments, all the buffers used for NA extraction have the same pH. In some embodiments, none of the buffers used for NA extraction have the same pH. In some embodiments, 1 or more buffers used for NA extraction have the same pH. Non-limiting examples of buffers that may be used in cfNA extraction include lysis buffer, binding buffer, wash buffer, and elution buffer.

In some embodiments, the buffers used for cfNA extraction have a pH that is basic. In some embodiments, the buffers used for cfNA extraction have a pH that is slightly acidic. In some embodiments, the buffers used for cfNA extraction have a pH that is neutral. In some embodiments, the pH range of the buffers used for cfNA extraction have a pH from about 6 to about 9. In some embodiments, all the buffers used for cfNA extraction have the same pH. In some embodiments, none of the buffers used for cfNA extraction have the same pH. In some embodiments, 1 or more buffers used for cfNA extraction have the same pH. Non-limiting examples of buffers that may be used in cfNA extraction include lysis buffer, binding buffer, wash buffer, and elution buffer.

In some embodiments, the eluted NA fragments are analyzed. In some embodiments, the NA fragments are analyzed using a nucleic acid-based detection assay. In some embodiments, the eluted cfNA fragments are analyzed. In some embodiments, the cfNA fragments are analyzed using a nucleic acid-based detection assay. In some embodiments, the nucleic acid-based detection assay comprises qPCR, gel electrophoresis (including for e.g., Northern or Southern blot), immunochemistry, in situ hybridization such as fluorescent in situ hybridization (FISH), cytochemistry, or sequencing. In some embodiments, the sequencing technique comprises next generation sequencing. In some embodiments, the methods involve a hybridization assay such as fluorogenic qPCR (e.g., TaqMan™ or SYBR green), which involves a nucleic acid amplification reaction with a specific primer pair, and hybridization of the amplified nucleic acid probes comprising a detectable moiety or molecule that is specific to a predetermined nucleic acid sequence. In some embodiments, the electrophoresis is automated. In some embodiments, the electrophoresis utilizes a TapeStation system.

In some embodiments, the eluted NA fragments contain no less than 50% non-canonical fragments. In some embodiments, the eluted NA fragments contain no less than 55% non-canonical fragments. In some embodiments, the eluted NA fragments contain no less than 60% non-canonical fragments. In some embodiments, the eluted NA fragments contain no less than 65% non-canonical fragments. In some embodiments, the eluted NA fragments contain no less than 70% non-canonical fragments. In some embodiments, the eluted NA fragments contain at least 70% non-canonical fragments.

In some embodiments, the eluted cfNA fragments contain no less than 50% non-canonical fragments. In some embodiments, the eluted cfNA fragments contain no less than 55% non-canonical fragments. In some embodiments, the eluted cfNA fragments contain no less than 60% non-canonical fragments. In some embodiments, the eluted cfNA fragments contain no less than 65% non-canonical fragments. In some embodiments, the eluted cfNA fragments contain no less than 70% non-canonical fragments. In some embodiments, the eluted cfNA fragments contain at least 70% non-canonical fragments.

In some embodiments, the NA fragments are analyzed to identify a disorder, a disease, a condition, or any combination thereof. In some embodiments, the NA fragments are analyzed to identify more than one disorder, disease, condition, or any combination thereof. Non-limiting examples of conditions indicated by NA includes cancer, systemic lupus erythematosus, and rheumatoid arthritis. In some embodiments, the NA fragments are analyzed to identify overabundance of a cell type (e.g. via identification of a sequence present in an overexpressed gene characteristic of the cell type). The overabundance can comprise inflammatory cells or pathological cells.

In some embodiments, the cfNA fragments are analyzed to identify a disorder, a disease, a condition, or any combination thereof. In some embodiments, the cfNA fragments are analyzed to identify more than one disorder, disease, condition, or any combination thereof. Non-limiting examples of conditions indicated by cfNA includes cancer, systemic lupus erythematosus, and rheumatoid arthritis. In some embodiments, the cfNA fragments are analyzed to identify overabundance of a cell type (e.g. via identification of a sequence present in an overexpressed gene characteristic of the cell type). The overabundance can comprise inflammatory cells or pathological cells.

In some embodiments, the disorder, disease, or condition is an autoimmune disorder, disease, or condition. As used herein, the terms “autoimmune disorder”, “autoimmune disease”, or “autoimmune condition” can be used interchangeably and refer to any disorder, disease, or condition in which the body produces an immunogenic (e.g., immune system) response to some constituent of its own tissue. In other words the immune system loses its ability to recognize some tissue or system within the body as “self” and targets and attacks it as if it were foreign. Autoimmune disorders, diseases, or conditions can be classified into those in which predominantly one organ is affected (e.g., hemolytic anemia and anti-immune thyroiditis), and those in which the autoimmune disorder, disease, or condition process is diffused through many tissues (e.g., systemic lupus erythematosus). Examples of autoimmune disorders, diseases, or conditions include, but are not limited to, rheumatoid arthritis, multiple sclerosis, lupus erythematosis, myasthenia gravis, scleroderma, Crohn's disease, ulcerative colitis, Hashimoto's disease, Graves' disease, Sjogren's syndrome, polyendocrine failure, vitiligo, peripheral neuropathy, autoimmnune polyglandular syndrome type I, acute glomerulonephritis, Addison's disease, adult-onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barré syndrome, hemochromatosis, Henoch-Schonlein purpura, idiopathic IgA nephropathy, insulin-dependent diabetes mellitus (IDDM), juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome, inflammatory bowel disease, osteoarthritis and thyroiditis.

### EXAMPLES

### Example 1. Chromatin Capture of cfDNA for Increased Non-canonical cfDNA Yield

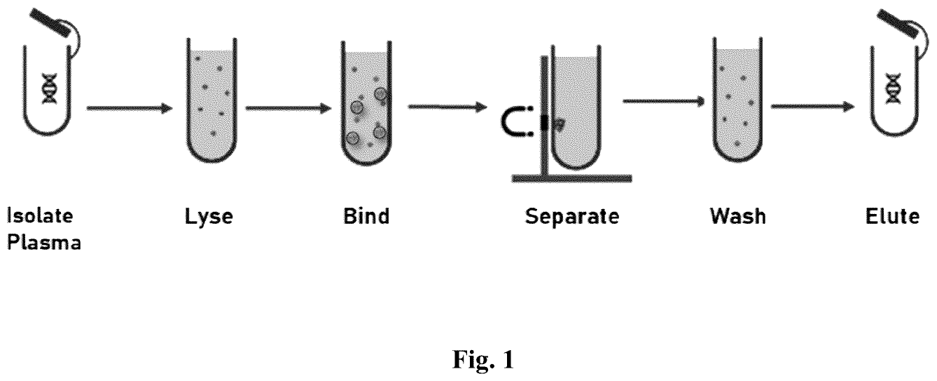

The workflow for cfNA (e.g. cfDNA) isolation broadly involved isolation of plasma from whole blood, lysis/digestion of plasma, binding nucleic acids in plasma to a solid support, washing the nucleic acids while bound to a solid support and eluting the nucleic acids from the solid support as shown in FIG. 1.

First, plasma was separated from whole blood using double spin centrifugation (e.g. by collection of blood in a blood collection tube such as a STRECK tube by a venipuncture blood draw). The blood collection tube was first centrifuged for 20 minutes at 300 xg at 4° C. with an acceleration setting of 9 and a deceleration setting of 4 to remove red blood cells. The supernatant layer was transferred from the blood collection tube to a 15 ml conical tube or 5 mL flip cap tube. The 15 ml conical tube or 5 mL flip cap tube was then centrifuged for 10 minutes at 5000xg at 4° C. with an acceleration and deceleration setting of 9 to separate platelets and white blood cells from plasma. The supernatant (e.g., plasma) layer was then transferred from the 15 ml conical tube to 2 mL aliquot tubes or 5 mL flip cap tube.

Plasma samples were then subjected to proteolytic digestion. One mL plasma was digested with 50 μL lysis buffer at pH 8.0 comprising guanidinium isothiocyanate at a final concentration of 0.26 M and 20 μL proteinase K at 58° C. for 30 minutes to help breakdown nucleases and release the cfNA (e.g. cfDNA) from native protective structures.

As this plasma contained canonical and non-canonical cfNA (e. g cfDNA) fragments (e.g. canonical short fragments approximately 170 nucleotides in length and non-canonical cfDNA fragments of approximately 300 nucleotides in length or longer), the nucleic acids were subjected to a first anionic capture operation. The digested plasma containing canonical and non-canonical cfNAs was mixed with 7.5 μL magnetic silica nanoparticles (e.g. those synthesized as described in Liu et al. J Chromatogr B Analyt Technol Biomed Life Sci. 2022 May 30; 1199:123236 or Sun et al. Angew Chem Int Ed Engl. 2004 February 1; 43(5):597-601, both of which are incorporated herein by reference in their entireties) and 500 μL binding solution at pH 7.8 plus 125 μL isopropanol for a final concentration of 1.8 M guanidinium isothiocyanate, 7.3% isopropanol, and 3.2-3.3% Brij- 58. The plasma and nanoparticles were incubated for 30 minutes at room temperature on a tube rotator set to a level 15 of speed.

After rotating, the tubes were set on a magnetic rack and the supernatant removed leaving the beads behind. The beads were then washed with 1 mL wash buffer at pH 8.1 comprising 2.5 M guanidinium isothiocyanate, 15% ethanol, and 3% nonionic polyoxyethylene surfactant. The tubes were again set on a magnetic rack and the supernatant removed leaving the nanoparticles behind. The beads were then washed two more times for a total of three washes with 1 mL 80% ethanol at pH 8.0. Following the final wash operation, the beads were dried. After drying, 32 μL of Tris-based elution buffer at pH 9.0 was added to the tube containing the dried beads allowing for complete rehydration and dissociation of the cfDNA from the nanoparticles.

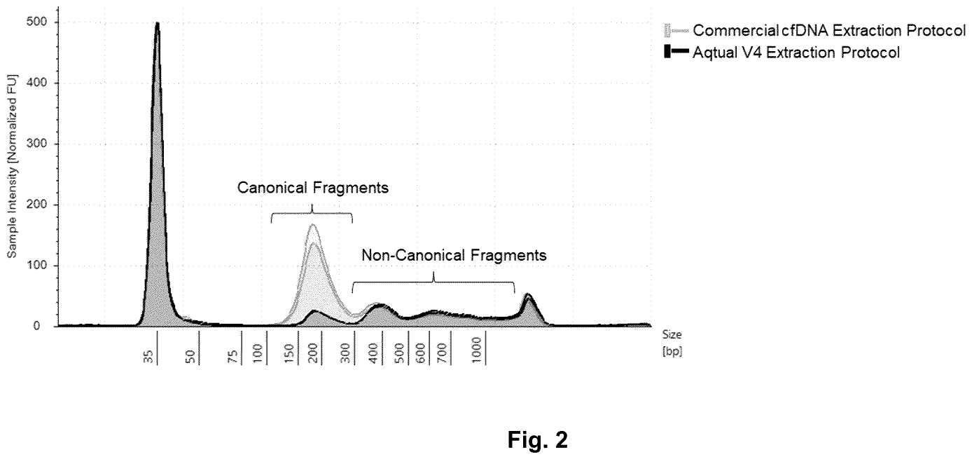

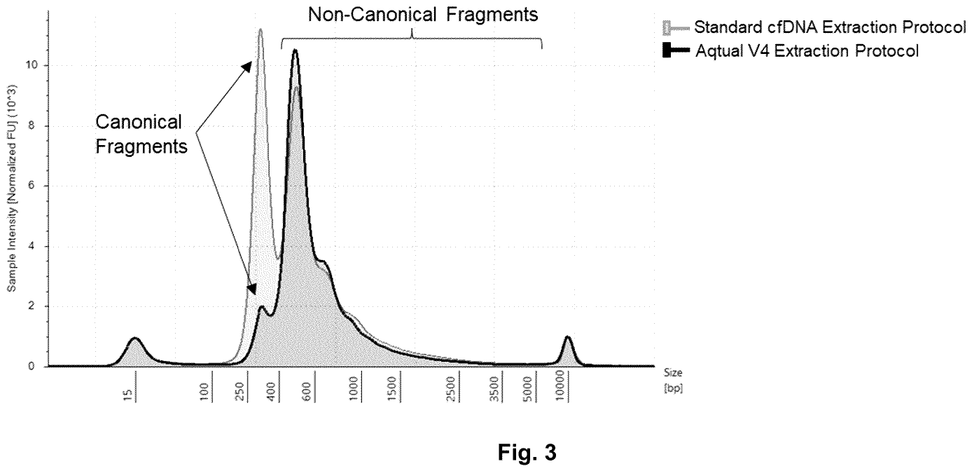

The isolated and purified non-canonical cfNA (e.g. cfDNA) was quantified and lengths verified using Agilent TapeStation 4200 Cell-Free DNA ScreenTape Assay and compared to commercial cfDNA extraction protocols (FIG. 2). The samples were then processed through library preparation (which removed additional canonical fragments through solid-phase reversible immobilization (SPRI) clean-up operations after ligation and amplification). Following amplification, the libraries were diluted 1:5 and 2 μL of the dilution was used as input into the Agilent TapeStation 4200 High Sensitivity D5000 ScreenTape assay and compared to commercial cfDNA extraction protocols.

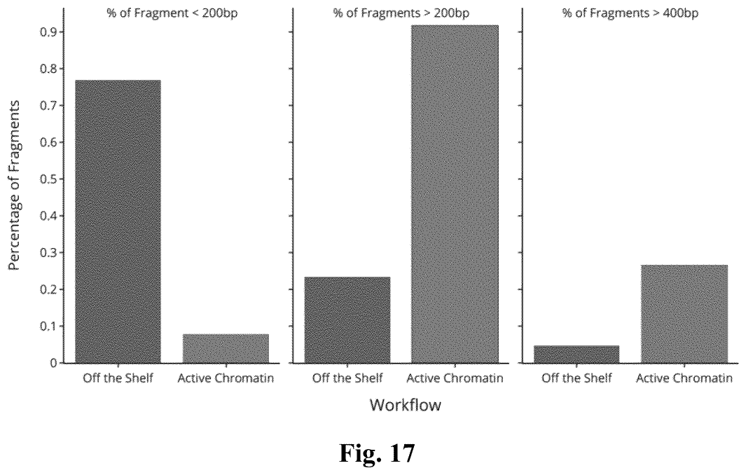

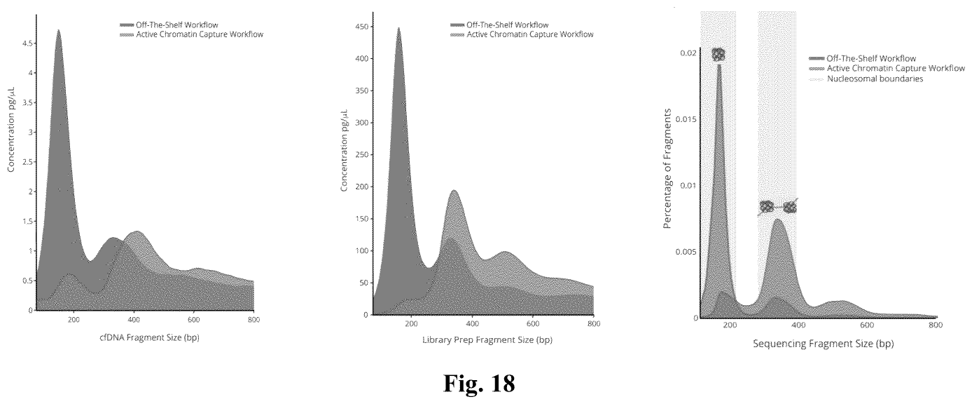

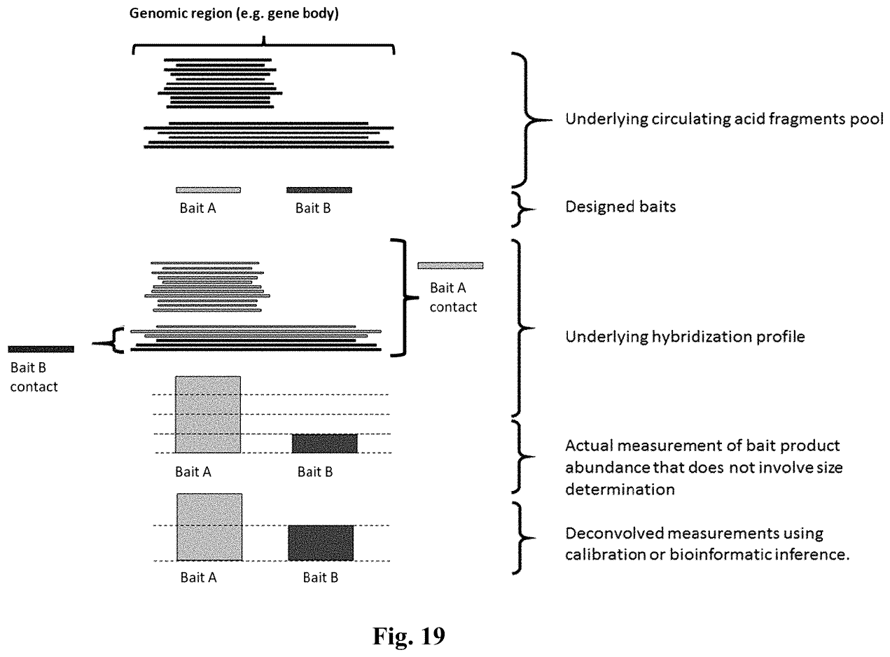

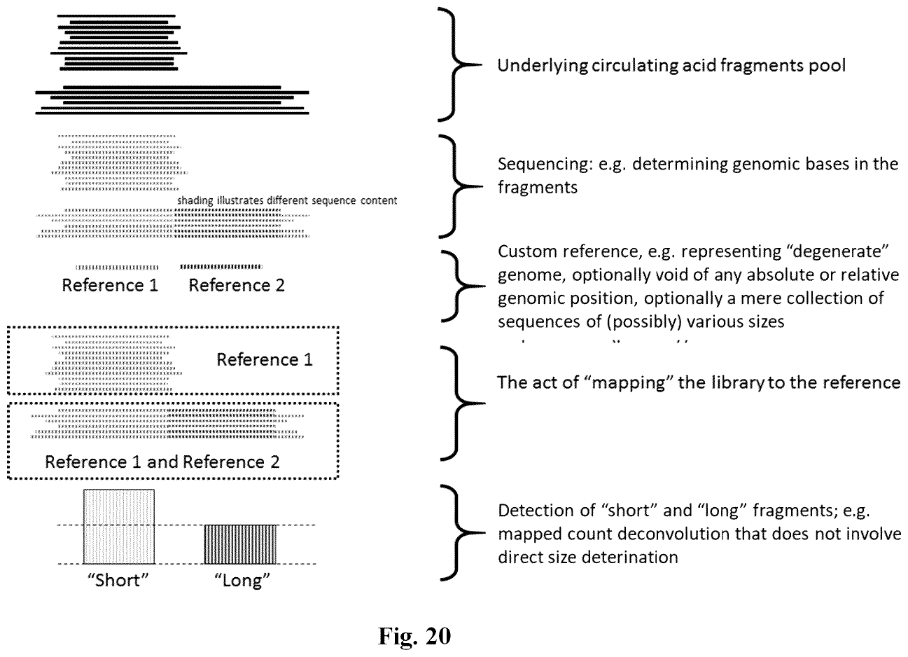

The results of the comparison to commercial (“standard”) cfNA (e.g. cfDNA) extraction protocols is shown in FIGS. 17 and 18, which shows abundance of fragments (in terms of fluorescence units) versus size for each protocol. Overall, the standard cfNA (e.g. cfDNA) extraction protocol resulted in median fragment length distribution of a dominant peak at approximately 167 nucleotides in length and a smaller second peak at approximately 334 nucleotides in length representing the mono- and di-nucleosome profile, respectively. The standard cfNA extraction protocol resulted in approximately 77% of fragments less than 200 bp. In contrast, the workflow disclosed herein produced a shifted fragment length distribution with the first peak approximately 10 nucleotides longer than the first peak of the standard cfNA extraction protocol as well as 8% of fragments less than 200 bp. Compared to standard cfNA extraction protocol capture kits, the workflow disclosed herein resulted in a 3.9-to 5.5-fold increase in the amount of longer (≥200 bp and >400 bp) cfDNA fragments (FIGS. 17 and 18), which include the cfDNA_{ac} fragment population. These cfDNA_{ac} fragments comprise active chromatin complexes bound to regulatory regions-gene promoters, enhancers, and transcription start sites-as well as gene bodies.

### Example 2. Comparison of Buffers on cfDNA Yield

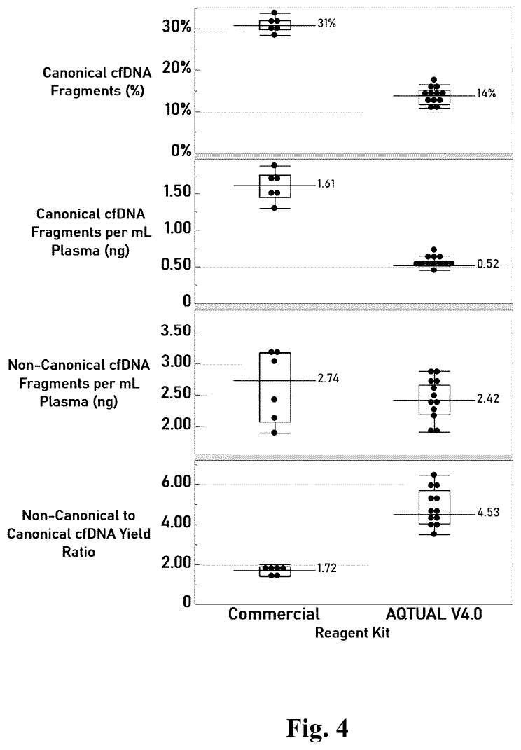

Commercial extraction buffers and the buffers disclosed herein were used in side-by-side experiments to assess the impact on enrichment of particular length populations of cfNA (e.g. cfDNA). The results showed that use the commercial extraction buffers resulted in a higher percentage of canonical fragments as compared to use of the buffers disclosed herein. Use of the commercial extraction buffers also yielded a higher concentration in nanograms of canonical cfNA (e.g. cfDNA) per mL of plasma. The two extraction buffer sets yielded similar concentrations in nanograms of non-canonical cfNA (e.g. cfDNA) fragment per mL of plasma. However, due to the low amount of canonical cfNA in protocols that used the buffers disclosed herein, the non-canonical to canonical cfNA yield ratio was much higher with protocols that used the buffers disclosed herein as compared to the protocols that used the commercial buffers (FIG. 4).

### Example 3. Regulatory-chromatin Length Fragments in Cell-free Nucleic Acids Recapitulate ChIP-Seq Signals

### Procedure/Results

Samples from five healthy individuals were collected across 14 timepoints and processed using the cell-free nucleic acid (e.g. cfDNA) extraction protocol described in Example 1. Samples from each individual (n=14) were sequenced on a NovaSeq 6000 sequencer using the S4 Reagent Kit v1.5 for 200 cycles. Reads were aligned to the human genome (hg19) and converted to fragments taking into consideration the entire fragment size from the start of read 1 to the end of read 2. Fragments were separated into nucleosomal cfDNA (e.g. cfDNA_{nuc}, fragments<210 nucleotides in length and 335-400 nucleotides in length) and regulatory or active chromatin cfDNA fragments (e.g. cfDNA_{ac}, fragments 211-334 nucleotides in length and >400 nucleotides in length) based on fragment size bins and converted to genome-wide signals (bigWig files). The deconvolution of cfNA (e.g. cfDNA) signal derived from active chromatin was accomplished by unsupervised clustering of fragment count profiles for different fragment size groups or bins. After deconvolution, approximately 52% of fragments were defined as cfDNA_{ac}.

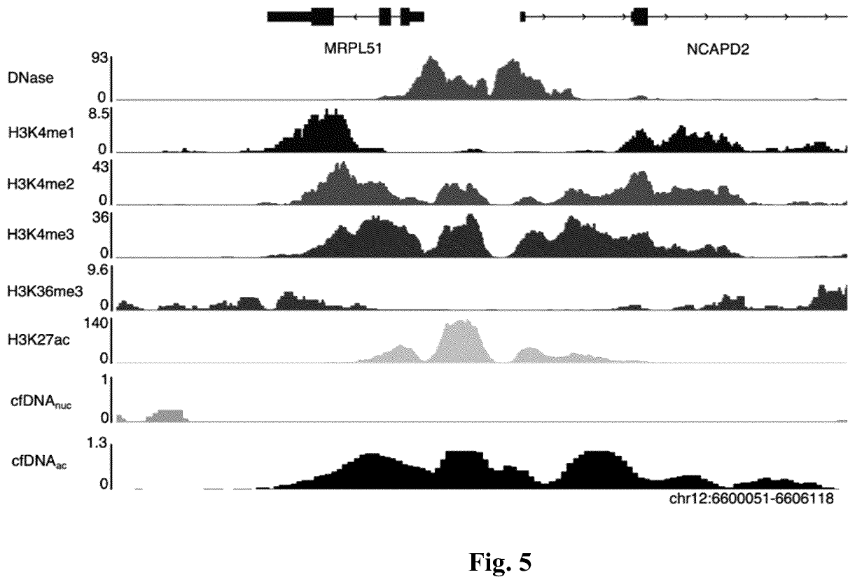

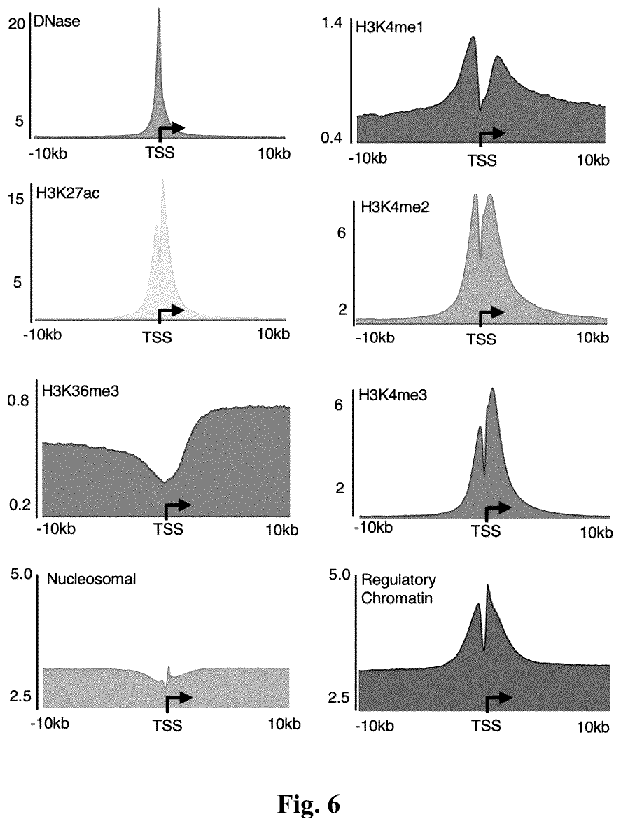

The cfDNA_{ac} fragment distribution was compared with ENCODE mappings at active promoters. The cfDNA_{ac} fragment distribution overlaps with ENCODE mappings for several active promoter regions (FIG. 5). In contrast, the cfDNA_{nuc} fragments are not enriched in these regions. These results suggest that the cfDNA_{ac} fragments are derived from transcription factor (TF)-bound complexes within the genome's regulatory regions and are protected from rapid degradation. The cfDNA_{ac} fragments also show an enrichment of signal around the transcription start sites (TSS) identified by ChIP-seq of documented histone modifications in order to identify promoter regions (FIG. 6). computeMatrix (deepTools) was used to compute the signal distribution relative to transcription start sites (TSSs) from the updated Eukaryotic Promoter Database (EPDnew) with a +/−10 kilobase (kb) flank and a bin/window size of 50.

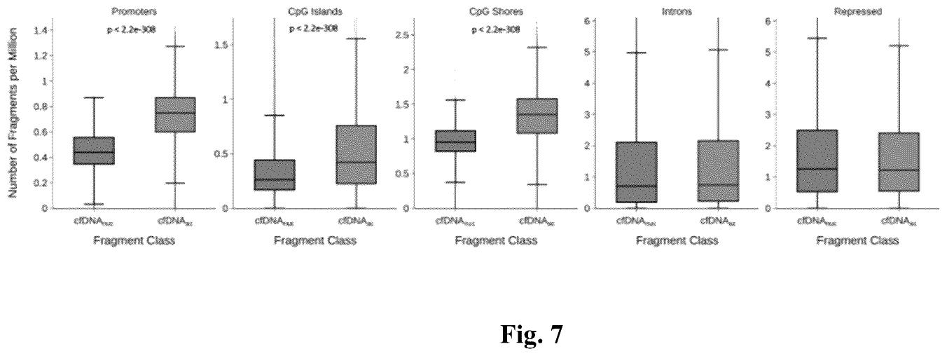

Extending the analysis across the genome, cfDNA_{ac} fragments are enriched in CpG islands and CpG shores compared to cfDNA_{nuc} fragments (FIG. 7). These data also indicate that enrichment was not observed in intronic and repressed regions across the genome for cfDNA_{ac}. In addition a correlation was observed between the regions enriched in cfDNA_{ac} fragments and regions comprising markers of active chromatin structure.

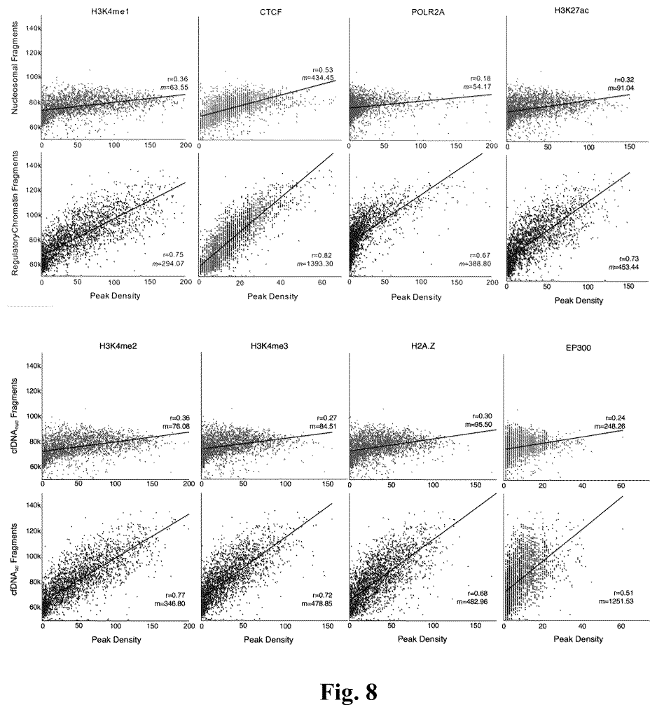

Correlations of the nucleosomal and regulatory-chromatin fragments were compared to the peak densities of the GM12878 cell line narrow peak calls for histone H3K4mel (histone H3 lysine 4 monomethyl), CTCF (CCCTC-binding factor), POLR2A (DNA-directed RNA polymerase II subunit RPB1), and H3K27ac (histone H3 lysine 27 acetylation) (FIG. 8). The sum of nucleosomal and regulatory-chromatin fragments for each of the five individuals was calculated for every 1 Megabase (Mb) window across the genome. The number of fragments within each 1 Mb window were correlated to the number of narrow peaks in H3K4me1, CTCF, POL2RA, H3K27ac, H3K4me2, H3K4me3, H2A.Z, and EP300. In contrast to nucleosomal fragments, analysis of the signal distribution of the regulatory-chromatin fragments showed an enrichment of signal that surrounded the transcription start sites (TSS) which is similar to ChIP-seq of histone modifications documented as associated with promoter regions. A strong correlation was observed in regulatory-chromatin fragments and not nucleosomal fragments for: H3K4me1,POLR2A, H3K27ac, H3K4me2, H3K4me3 and H2A.Z. A mild correlation was observed for EP300. Nucleosomal fragments did not show a strong correlation with any of these regulatory elements.

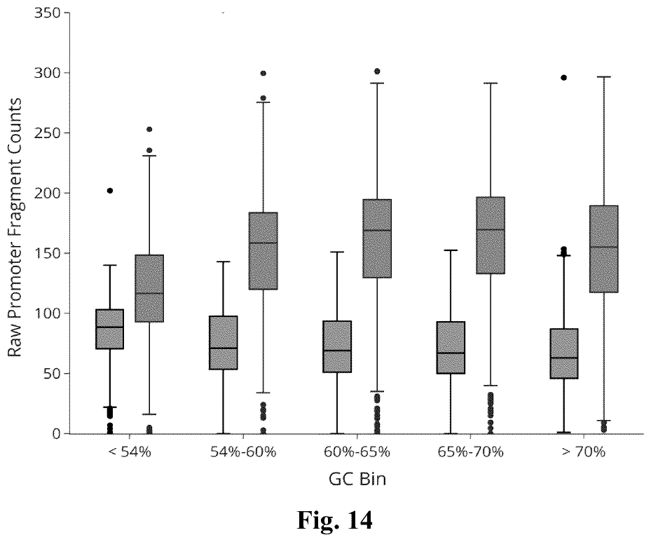

To investigate whether the higher GC % played a part in enriching fragments, the FANTOM5 TSS locations were stratified by GC % (0-20^{th} percentile, 20^{th}-40^{th} percentile, 40^{th}-60^{th} percentile, 60^{th}-80^{th} percentile, and 80^{th}-100^{th} percentile) and compared to raw fragment counts at these locations (FIG. 14). Regardless of GC bin, there was a significant enrichment of active chromatin relative to nucleosomal fragments.

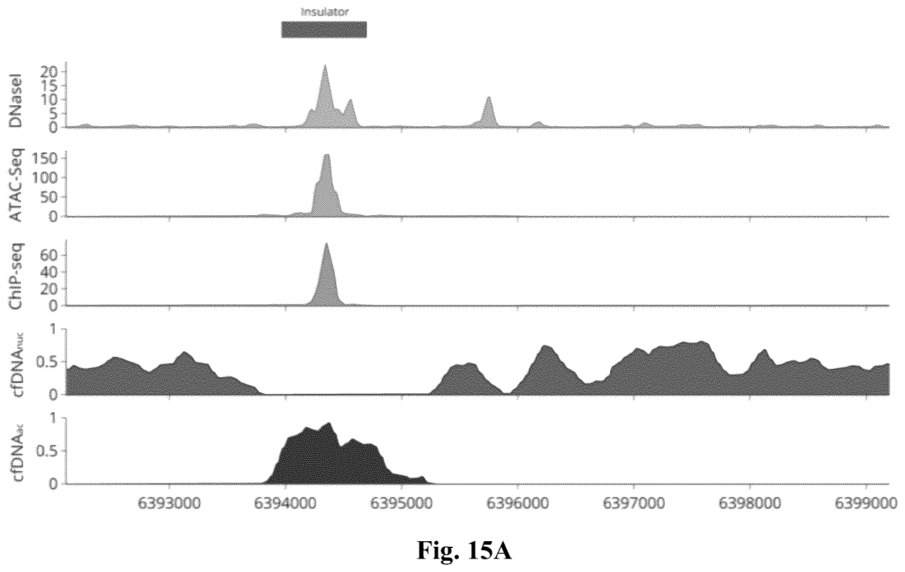

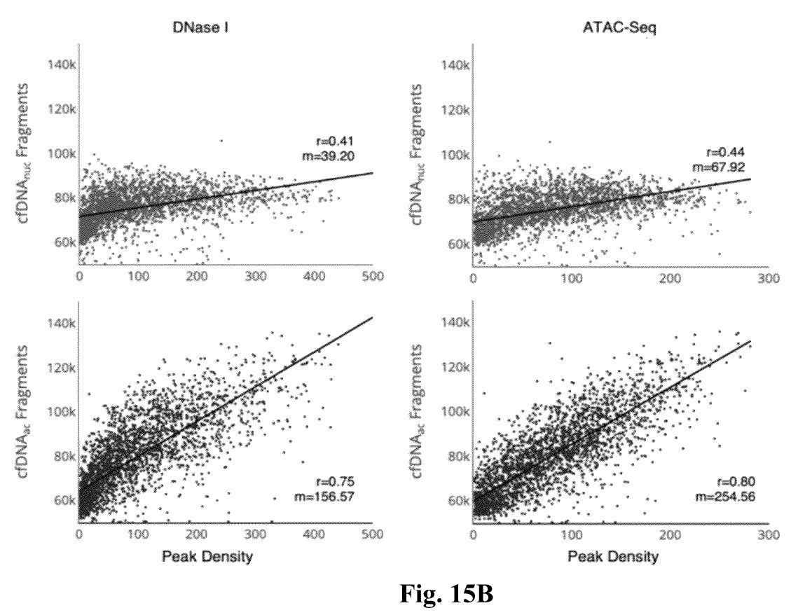

The correlation between the cfDNA_{ac} fragment abundance and H3K4mel narrow peak densities in 1 Mb (megabase) segments showed a strong Pearson correlation with r=0.75 (p<2.2e−308). Similar correlations to that between cfDNA_{ac} and H3K4mel were observed for other histone markers, e.g. H3K4me2 (r=0.76, p<2.2e−308) and H3K4me3 (r=0.72, p<2.2e−308) (FIG. 8). In contrast, the cfDNA_{nuc} fragments do not show meaningful correlation with histone markers analyzed. With respect to cfDNA_{nuc}, results indicate that cfDNA_{nuc} fragments can footprint CTCF (FIG. 8) with a moderate correlation (r=0.53, p=2.3e−153). In contrast, the cfDNA_{ac} fragments showed a higher correlation (r=0.82, p<2.2e−308) with CTCF peaks. Regulatory-active chromatin fragments also show a significant correlation with H3K27ac (FIG. 8; r=0.73, p<2.2e−308), a histone modification documented as associated with enhancers, and with POLR2A (FIG. 8; r=0.67, p<2.2e−308), which encodes for the largest subunit of RNA polymerase II. Similarly, other regions associated with enhancers (e.g. H2A.Z, FIG. 8; r=0.68, p<2.2e−308) and EP300 narrow peak densities (FIG. 8; r=0.51, p=3.2e−147) also show positive correlations with cfDNA_{ac} fragment levels. cfDNA_{ac} and cfDNA_{nuc} was also evaluated for correlation with GM12878 DNase-I and ATAC-Seq signals (FIGS. 15A and 15B). The number of cfDNA_{ac} fragments strongly correlate with both DNase-I (r=0.75, p<2.2e−308) and ATAC-Seq peak densities (r=0.80, p<2.2e−308), whereas cfDNA_{nuc} did not correlate with either.

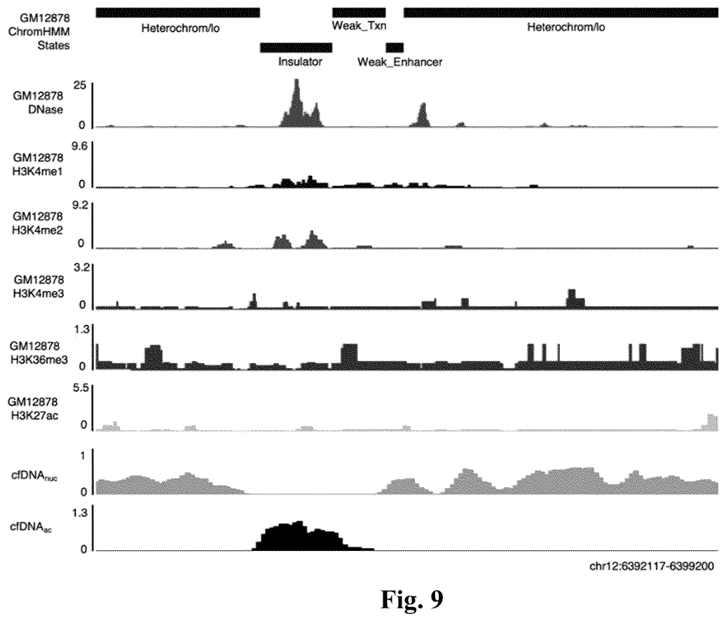

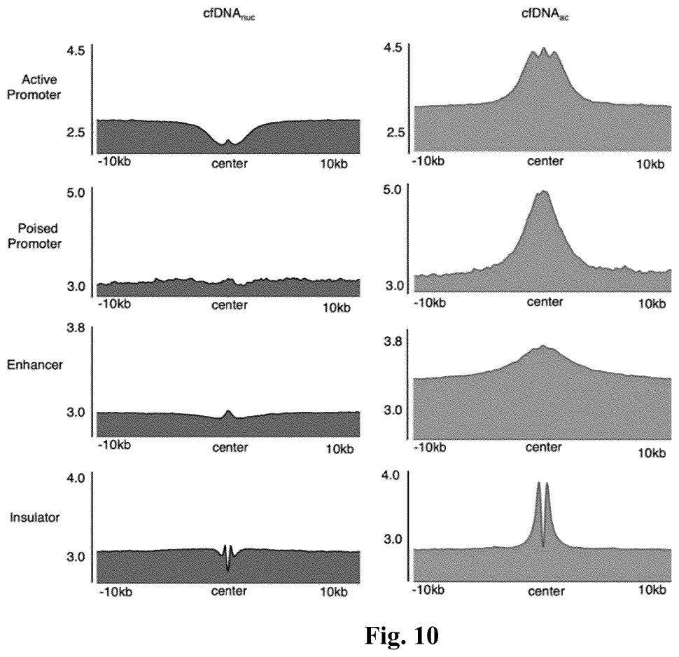

Comparison of the cfDNA_{ac} fragment distribution with other ENCODE categories also shows an enrichment of these fragments at insulator regions (FIG. 9). Utilizing ENCODE ChromHMM annotations, we found a strong enrichment of cfDNA_{ac} fragments across different chromatin states (FIG. 10).

### Connection to Circadian Genes

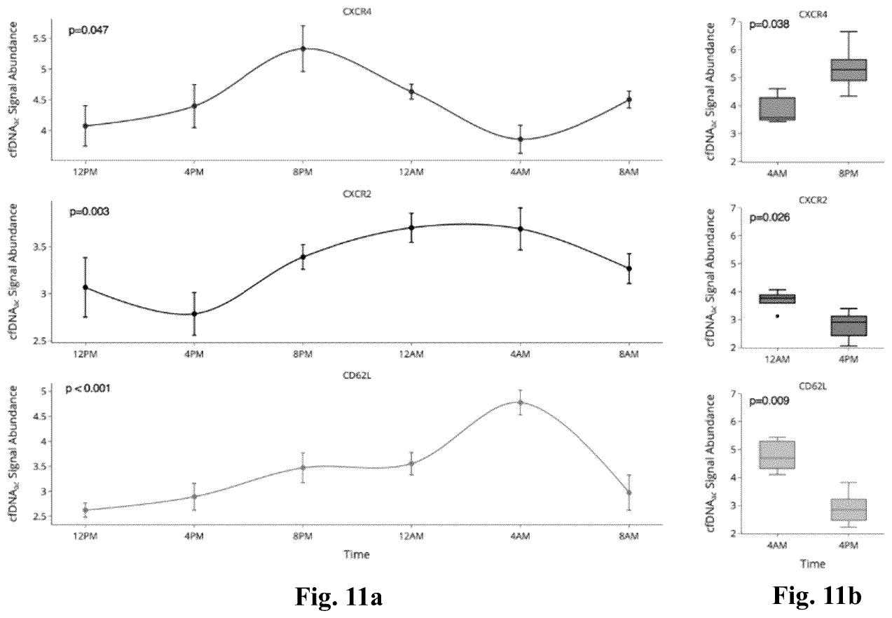

Circadian genes and their related transcription factors have been shown to regulate numerous immune activities. Specifically, neutrophils have been documented to follow a diurnal pattern, where neutrophil markers CXCR4, CXCR2, and SELL (CD62L) have opposing circadian oscillations as they are released from the bone marrow and egress from the blood. Since there is a strong correlation of cfDNA_{ac} with histone modifications—which are indicative of active promoters—an analysis was performed to determine if cfDNA ac can be used to detect the diurnal patterns of these highly expressed circadian genes found in whole blood. The analysis focused on circadian-specific markers with varying expression levels in whole blood (ranging from 1.61 to 1300 transcripts per million (TPM, with a median 187.73), as defined by GTEx (Genotype-Tissue Expression Project). The abundance of the cfDNA_{ac} signal at promoters indicates significant circadian oscillations for CXCR4 (p=0.047), CXCR2 (p=0.003), and CD62L (p<0.001; FIG. 11a), three neutrophil-specific circadian markers. CXCR4 has an opposing oscillation to CXCR2 and CD62L. CXCR4 shows a decrease in cfDNA_{ac} levels while both CXCR2 and CD62L show higher signal abundance early in the morning. Additionally, the difference in cfDNA_{ac} signal was statistically significant between early morning and evening for CXCR4 (p=0.038), CXCR2 (p=0.026), and CD62L (p=0.009; FIG. 11b). The cfDNA_{ac} signal for three known regulators of circadian rhythm-CRY1 (p=0.009), TIMELESS (p=0.007), and PER1 (p=0.011)—also showed significant variations between early morning and evening. Consistent with their known interaction, CRY1, PER1, and TIMELESS exhibit similar circadian patterns. However, statistical significance was observed only for CRY1 (p=0.011) and TIMELESS (p=0.019).

### Fragment Correlation With Expression

Given that cfDNA_{ac} abundance can capture the transcriptional oscillations in highly expressed circadian genes, coupled with the observed correlations with regulatory elements mapped in ENCODE, an analysis was performed to determine if the cfDNA_{ac} fragments can be utilized to measure (or serve as a proxy measurement of) gene expression at the transcriptional level. As disclosed herein (e.g. FIG. 8), the cfDNA_{ac}—and not the cfDNA_{nuc}—fragment signal correlated with POLR2A ChIP-Seq densities.

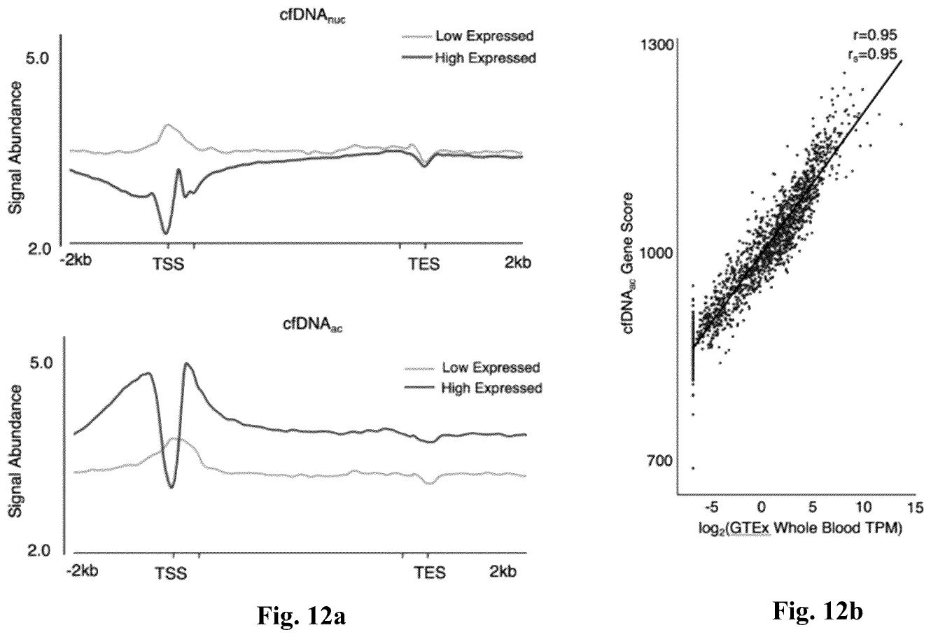

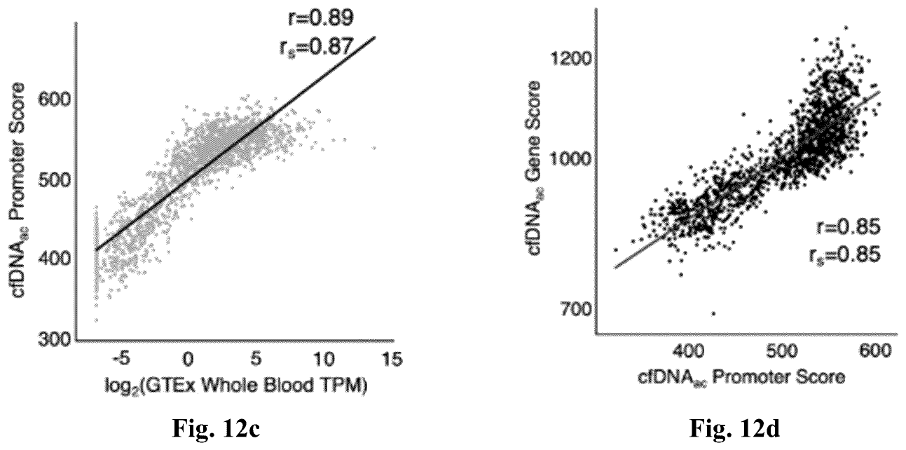

Utilizing expression levels defined by GTEx, the read-depth patterns of the cfDNA_{nuc} and cfDNA_{ac} fragments were compared across gene bodies stratified by highest (top 10%) and lowest expressed (bottom 10%) genes. The cfDNA_{nuc} signals show a coverage depletion around the TSS for highly expressed genes (FIG. 12a), which may be due to less dense nucleosome packaging. However, cfDNA_{ac} fragments show increased signals for highly expressed genes, both around the TSS and across the entire gene body (FIG. 12a), as these fragments may represent transcription factor-bound regions. This signal profile is consistent with promoter-proximal RNA polymerase II pausing.

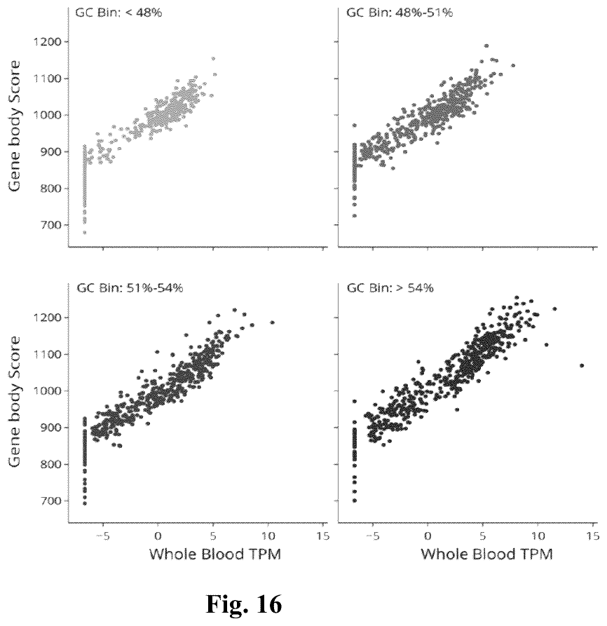

Assuming the hypothesis that cfDNA_{ac} fragments can be used to recapitulate gene expression levels is correct, correlation between the promoter and gene body scores and gene expression levels should be high. Whole blood RNA-sequencing expression levels were obtained from GTEx and compared to promoter and gene body scores obtained from cfDNA_{ac}. These cfDNA_{ac} fragment scores across both gene bodies (FIG. 12 b, r=0.95, p<2.2 e−308) and promoters (FIG. 12 c, r=0.89, p<2.2 e−308) highly correlate with GTEx whole blood TPM values. The signal at promoters exhibits a plateau for highly expressed genes (FIG. 12c), which can be to a coverage depletion in the nucleosome-depleted region (NDR). However, the gene body cfDNA_{ac} signal does not exhibit this plateau. The promoter and gene body cfDNA_{ac} signals at both regions are significantly correlated with one another (FIG. 12 d, r=0.85, p<2.2e−308), consistent with the expectation that the promoter and gene body are each bound by regulatory proteins, and therefore are independently associated with gene expression. The correlation by GC content was also assessed with each bin representing the 25^{th} percentile of the average GC content of the gene body. The correlation with GTEx whole blood gene expression remains consistent across all GC bins (FIG. 16).

The cfDNA extraction protocol (Example 1) yielded other populations of fragments that were not solely nucleosomal bound. Profiling these fragments showed they are coming from bound DNA-protein complexes associated with regulatory chromatin. Importantly, bound DNA-protein complexes were captured with an antibody-free assay.

### Example 4. Alternative Lysis Buffer Formulation

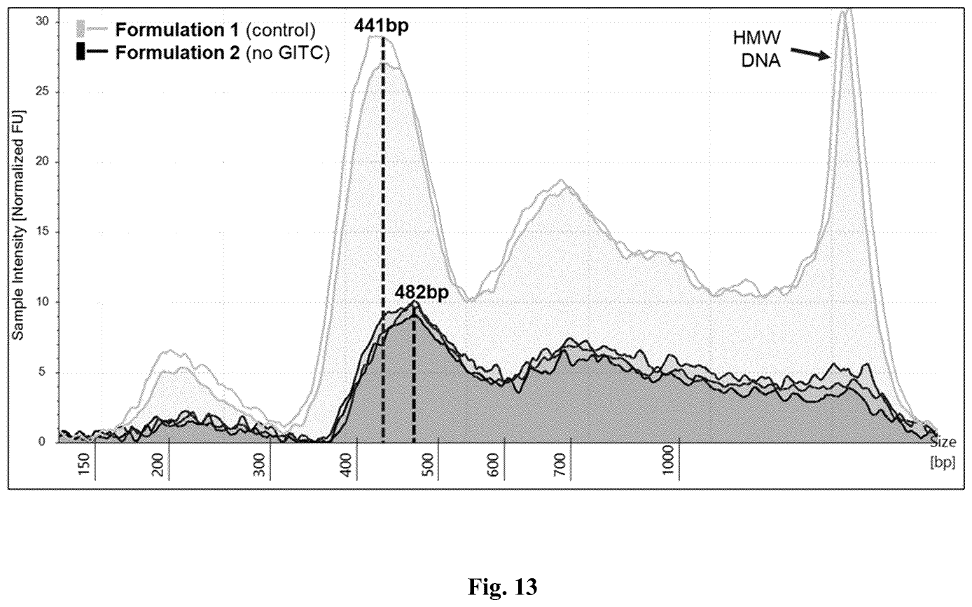

Two milliliters (mL) of human plasma previously separated from whole blood collected in a STRECK cell-free DNA (cfDNA) blood collection tube was processed using the cfNA (e.g. cfDNA) extraction protocol described in Example 1 with minor alterations. Briefly, 0.37 mg/mL Proteinase K was used to process the samples with two different lysis buffer formulations, Formulation 1 and Formulation 2. Both lysis buffer formulations contained 1.2 mM Tris HCl pH 8.0, 0.06 mM EDTA, and 10.4% Brij-58 but had different concentrations of guanidine isothiocyanate (GITC). Formulation 1 was comprised of 5.5 M GITC. Formulation 2 was comprised of 0 M GITC. 50 μL of lysis buffer per 1 mL plasma was used for formulation 1. 200 μL of lysis buffer per 1 mL plasma was used for formulation 2. After incubating the sample with the lysis buffer at 58° C. for 30 minutes, the samples were incubated for 30 minutes at room temperature with the magnetic silica nanoparticles using equivalent binding conditions. Following binding, the samples were processed with identical wash and elution conditions. Fragment length and yields were determined by running equal volumes of cfDNA eluate (2 microliters (μL)) on Agilent's Cell-Free DNA TapeStation assay (FIG. 13).

Samples processed with the Formulation 2 lysis buffer (without GITC) produced a median 69% decrease in total yield with a ~41 base pair (bp) increase in length for the second peak (median length =482 bp; FIG. 13) compared to Formulation 1 (control). Samples processed with the Formulation 2 lysis buffer (without GITC) did not contain the peak that usually exceeds 10,000 nucleotides in length correlating to high molecular weight (HMW) fragments. These HMW fragments are thought to be intact genomic copies of DNA released from leukocytes and carried over from plasma separation into extraction. The lack of HMW fragments in the samples processed with the Formulation 2 lysis buffer indicates that GITC is required to lyse cells and lysis without GITC produces extracts free of HMW DNA.

Standard cfDNA protocols follow the same workflow of digestion, binding, washes, and elution where digestion occurs during lysis incubation using proteinase K in the presence of chaotropic salts (such as GITC) and heat to release cfDNA from histone protein complexes. As such, it was hypothesized that in the absence of GITC, very little cfDNA release would be released. However, Formulation 2 was able to extract a population of longer, non-mononucleosomal sized fragments, which may represent regulatory chromatin fragments, with significant depletion of mononucleosomal sized fragments (~170 bp; FIG. 13, first peak). Although the decrease in total yield for samples processed with Formulation 2 was lower than standard protocols, a no GITC lysis buffer may allow for selective extraction of regulatory chromatin fragments without contamination from nucleosomal fragments.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-93. (canceled)

94. A method, comprising:
(a) providing a plurality of cell-free nucleic acid (cfNA) fragments comprising cfNA fragments derived from active chromatin and cfNA fragments derived from inactive chromatin;
(b) contacting said plurality of cfNA fragments with an anionic solid surface in a presence of a chaotropic agent, wherein, thereby coupling said cfNA fragments derived from active chromatin with said anionic solid surface;
(c) removing said cfNA fragments derived from inactive chromatin from said cfNA fragments derived from active chromatin coupled to said anionic surface; and
(d) detecting a gene in said cfNA fragments derived from active chromatin.

95. The method of claim 1, further comprising, after (c), decoupling said cfNA fragments derived from active chromatin from said anionic surface.

96. The method of claim 94, wherein said active chromatin comprises at least part of a promotor, an insulator, a transcriptional start site, a DNase hypersensitive site, a DNAse-I hypersensitive site, a Pol II pausing site, a first exon, or an intron to exon boundary, or any combination thereof.

97. The method of claim 94, wherein (b) further comprises contacting said plurality of cfNA fragments in a presence of an alcohol.

98. The method of claim 97, wherein said alcohol is part of a solution and said solution comprises at least about 3.5% to at most about 11% alcohol.

99. The method of claim 94, wherein said chaotropic agent comprises a guanidinium salt.

100. The method of claim 94, wherein said chaotropic agent comprises urea.

101. The method of claim 94, wherein said chaotropic agent is at a concentration of bout 1.5 molar (M) to about 2.0 M.

102. The method of claim 94, wherein (c) comprises washing said anionic solid surface with a wash solution.

103. The method of claim 102, wherein said wash solution comprises alcohol.

104. The method of claim 102, wherein said wash solution displays a pH of greater than or equal to about 6.0.

105. The method of claim 102, wherein said wash solution comprises a nonionic surfactant.

106. The method of claim 94, wherein said anionic solid surface comprises silicon dioxide (SiO₂).

107. The method of claim 94, wherein said anionic solid surface is a surface of a particle.

108. The method of claim 107, wherein said particle has a diameter of about 200 nanometers (nm) to about 600 nm.

109. The method of claim 107, wherein said particle comprises a hydroxyl-derivatized surface layer and a ferric core.

110. The method of claim 94, wherein said plurality of cfNA fragments in (a) are part of a plasma sample.
